(19) United States
(12) Patent Application Publication
(10) Pub. No.: US 2026/0000716 A1
(43) Pub. Date: Jan. 1, 2026

(54) METHODS AND COMPOSITIONS FOR THE TREATMENT OF CANCER

(71) Applicant: Wisconsin Alumni Research Foundation, Madison, WI (US)

(72) Inventors: John-Demian Sauer, Madison, WI (US); Paul Sondel, Madison, WI (US); Alexander Rakhmilevich, Lake Mills, WI (US); Kijeong Lee, Madison, WI (US); Zachary Morrow, Madison, WI (US)

(21) Appl. No.: 19/215,947

(22) Filed: May 22, 2025

Related U.S. Application Data
(60) Provisional application No. 63/651,513, filed on May 24, 2024.

(51) Int. Cl. A61K 35/741; A61P 35/00; C07K 14/54; C07K 14/55; C07K 14/705; C12N 15/74

(52) U.S. Cl. CPC A61K 35/741; A61P 35/00; C07K 14/5434; C07K 14/55; C07K 14/70575; C12N 15/74

(57) ABSTRACT

Disclosed herein are genetically engineered Listeria monocytogenes strains and methods and compositions comprising the same for preventing, ameliorating, or treating cancer. In particular, the present technology relates to a genetically engineered L. monocytogenes bacterium, wherein the bacterium comprises a nucleic acid encoding IL-2, IL-12, or CD-40L, and methods of using said bacterium for preventing, ameliorating, or treating cancer.

### CROSS-REFERENCE TO RELATED APPLICATIONS

This application claims the benefit of and priority to U.S. Provisional Patent Application No. 63/651,513, filed May 24, 2024, the entire contents of which are incorporated herein by reference.

### SEQUENCE LISTING

The instant application contains a Sequence Listing which has been submitted electronically in XML format and is hereby incorporated by reference in its entirety. Said XML copy, created on Mar. 18, 2025, is named 032026-1554_SL.xml and is 37,528 bytes in size.

### TECHNICAL FIELD

The present technology relates generally to genetically engineered Listeria monocytogenes and methods and compositions comprising the same for preventing, ameliorating, or treating cancer. In particular, the present technology relates to a genetically engineered L. monocytogenes bacterium, wherein the bacterium comprises a nucleic acid comprising a sequence encoding IL-2, IL-12, or CD-40L, and methods of using said bacterium for preventing, ameliorating, or treating cancer.

### BACKGROUND

The following description is provided to assist the understanding of the reader. None of the information provided or references cited is admitted to be prior art to the compositions and methods disclosed herein.

The treatment of cancer is a pressing public health challenge. High dose cytokines have been deployed for cancer therapy since the late 1980's. However, challenges including systemic toxicity, frequency of treatment, and poor drug-like properties have limited their use despite some successes. Numerous strategies to overcome these challenges have been attempted, yet few have been successful. Accordingly, there is a need for new methods and compositions to deliver targeted cytokine therapies while minimizing undesirable aspects.

Attenuated L. monocytogenes has previously been employed in clinical trials as a vaccine due to its ability to stimulate a CD8+ T-cell response and engineering capacity to express tumor-specific antigens. In preclinical studies, it was observed that L. monocytogenes accumulates in the tumor microenvironment while being cleared from healthy tissues, indicating its potential as a vector for administering treatments to cancerous tissues. Methods and compositions described herein are directed to the use of L. monocytogenes to deliver anti-tumor agents to tumor microenvironments.

### SUMMARY

In one aspect, the present disclosure provides a genetically engineered Listeria monocytogenes bacterium comprising a nucleic acid sequence encoding interleukin 2 (IL-2), interleukin 12 (IL-12), or Cluster of Differentiation 40 ligand (CD40L). In some embodiments, the nucleic acid sequence encodes IL-2. In some embodiments, the nucleic acid sequence encodes IL-12. In some embodiments, the nucleic acid sequence encodes CD40L. In some embodiments, the nucleic acid sequence further encodes a Listeria monocytogenes secretion signal. In some embodiments, the Listeria monocytogenes secretion signal comprises an ActA secretion signal. In some embodiments, the ActA secretion signal comprises amino acids 1 to 30 of the ActA protein or amino acids 1 to 100 of the ActA protein. In some embodiments, the ActA secretion signal comprises amino acids 1-100 of the ActA protein. In some embodiments, the Listeria monocytogenes secretion signal comprises the amino acid sequence set forth in SEQ ID NO: 9. In some embodiments, the nucleic acid sequence further comprises a 5′ untranslated region. In some embodiments, the 5′ untranslated region comprises the 5′ untranslated region of an hly gene. In some embodiments, the 5′ untranslated region comprises the nucleotide sequence set forth in SEQ ID NO: 11. In some embodiments, the nucleic acid sequence comprises SEQ ID NO: 10. In some embodiments, the nucleic acid sequence is integrated into the bacterial genome. In some embodiments, the nucleic acid sequence is integrated into the bacterial genome at the attBB′ site in the tRNA^{Arg} locus.

In one aspect, the present disclosure provides a composition for treating a cancer in a subject in need thereof comprising the genetically engineered Listeria monocytogenes bacterium of any one of the preceding embodiments.

In one aspect, the present disclosure provides a composition for expressing an immune potentiating agent in a tumor microenvironment of a tumor in a subject comprising the genetically engineered Listeria monocytogenes bacterium of any one of the preceding embodiments wherein the immune potentiating agent is selected from the group consisting of IL-2, IL-12, and CD40L.

In one aspect, the present disclosure provides a composition for expressing an immune potentiating agent in a tumor cell in a solid tumor in a subject comprising the genetically engineered Listeria monocytogenes bacterium of any one of the preceding embodiments, wherein the immune potentiating agent is selected from the group consisting of IL-2, IL-12, and CD40L.

In one aspect, the present disclosure provides a method of treating a cancer in a subject in need thereof comprising administering to the subject a composition comprising the genetically engineered Listeria monocytogenes bacterium of any one of the preceding embodiments. In some embodiments, the cancer comprises melanoma or neuroblastoma. In some embodiments, the cancer comprises one or more solid tumors. In some embodiments, the genetically engineered Listeria monocytogenes bacterium persists in the one or more solid tumors for about 1 day to about 3 weeks post administration. In some embodiments, the method is effective for reducing tumor volume or slowing tumor growth. In some embodiments, the composition is administered intravenously or intratumorally. In some embodiments, the genetically engineered Listeria monocytogenes bacterium comprises a nucleic acid sequence encoding IL-2. In some embodiments, the genetically engineered Listeria monocytogenes bacterium comprises a nucleic acid sequence encoding IL-12. In some embodiments, the genetically engineered Listeria monocytogenes bacterium comprises a nucleic acid sequence encoding CD40L. In some embodiments, the composition further comprises a pharmaceutically acceptable carrier. In some embodiments, the method further comprises administering to the subject an additional therapeutic agent. In some embodiments, the additional therapeutic agent is selected from the group consisting of radiation therapy and immune checkpoint blockade inhibitors. In some embodiments, the additional therapeutic agent is administered simultaneously, separately, or sequentially to the composition.

In one aspect, the present disclosure provides a method of expressing an immune potentiating agent in a tumor microenvironment of a solid tumor in a subject comprising administering to the subject a composition comprising the genetically engineered Listeria monocytogenes bacterium of any one of the preceding embodiments, wherein the immune potentiating agent is selected from the group consisting of IL-2, IL-12, and CD40L. In some embodiments, the cancer comprises melanoma or neuroblastoma. In some embodiments, the cancer comprises one or more solid tumors. In some embodiments, the genetically engineered Listeria monocytogenes bacterium persists in the one or more solid tumors for about 1 day to about 3 weeks post administration. In some embodiments, the method is effective for reducing tumor volume or slowing tumor growth. In some embodiments, the composition is administered intravenously or intratumorally. In some embodiments, the genetically engineered Listeria monocytogenes bacterium comprises a nucleic acid sequence encoding IL-2. In some embodiments, the genetically engineered Listeria monocytogenes bacterium comprises a nucleic acid sequence encoding IL-12. In some embodiments, the genetically engineered Listeria monocytogenes bacterium comprises a nucleic acid sequence encoding CD40L. In some embodiments, the composition further comprises a pharmaceutically acceptable carrier. In some embodiments, the method further comprises administering to the subject an additional therapeutic agent. In some embodiments, the additional therapeutic agent is selected from the group consisting of radiation therapy and immune checkpoint blockade inhibitors. In some embodiments, the additional therapeutic agent is administered simultaneously, separately, or sequentially to the composition.

In one aspect, the present disclosure provides a method of expressing an immune potentiating agent in a tumor cell in a solid tumor in a subject comprising administering to the subject a composition comprising the genetically engineered Listeria monocytogenes bacterium of any one of the preceding embodiments, wherein the immune potentiating agent is selected from the group consisting of IL-2, IL-12, and CD40L. In some embodiments, the cancer comprises melanoma or neuroblastoma. In some embodiments, the cancer comprises one or more solid tumors. In some embodiments, the genetically engineered Listeria monocytogenes bacterium persists in the one or more solid tumors for about 1 day to about 3 weeks post administration. In some embodiments, the method is effective for reducing tumor volume or slowing tumor growth. In some embodiments, the composition is administered intravenously or intratumorally. In some embodiments, the genetically engineered Listeria monocytogenes bacterium comprises a nucleic acid sequence encoding IL-2. In some embodiments, the genetically engineered Listeria monocytogenes bacterium comprises a nucleic acid sequence encoding IL-12. In some embodiments, the genetically engineered Listeria monocytogenes bacterium comprises a nucleic acid sequence encoding CD40L. In some embodiments, the composition further comprises a pharmaceutically acceptable carrier. In some embodiments, the method further comprises administering to the subject an additional therapeutic agent. In some embodiments, the additional therapeutic agent is selected from the group consisting of radiation therapy and immune checkpoint blockade inhibitors. In some embodiments, the additional therapeutic agent is administered simultaneously, separately, or sequentially to the composition.

### BRIEF DESCRIPTION OF THE DRAWINGS

The patent or application file contains at least one drawing executed in color. Copies of this patent or patent application publication with color drawing(s) will be provided by the Office upon request and payment of necessary fee.

FIGS. 1A-1D show L. monocytogenes engineered to express human IL-2. FIG. 1A is a diagram showing design of ActA-IL-2 fusion constructs constructed for genomic integration into L. monocytogenes. FIG. 1B is a schematic representation of L. monocytogenes expression construct, integration to the phage site of the genome, and expression of IL-2 fused to the n-terminus of ActA. FIG. 1C shows the results of an IL-2 ELISA performed on RPMI media used to grow L. monocytogenes expressing IL-2 for 1 hour at 37° C. FIG. 1D is a graph showing the CPM for CTLL-2 cells co-cultured in the presence of [³H]thymidine with 50% RPMI used to grow L. monocytogenes expressing IL-2 for 1 hour at 37° C. and then subjected to liquid scintillation counting.

FIGS. 2A-2F show that L. monocytogenes accumulates in the tumor microenvironment and can be engineered to express IL-2 in vivo. 2×10⁶ B78 cells were implanted into the flank of syngeneic mice. When tumors reached ˜300 mm³, 1×10⁷ LADD or LIS-IL-2 was injected IV or IT. Splenic (FIG. 2A), liver (FIG. 2B), and tumor (FIGS. 2C-2D) burdens were assessed on the indicated days. IL-2 was quantified in the tumor homogenates (FIG. 2E) and mouse serum (FIG. 2F) at the indicated timepoints. N=3 mice/group at each timepoint.

FIGS. 3A-3J show that OPT-LIS-IL-2 produces IL-2 and accumulates in tumors. FIG. 3A shows the design of an ActAN100-IL-2 fusion construct with hly 5′ UTR upstream of the fusion. OPT-LIS-IL-2 was grown for 1 hour in RPMI at 37° C. and clarified supernatants were tested for IL-2 by ELSIA (FIG. 3B) and CTLL-2 proliferation induction capacity (FIG. 3C). 2×10⁶ B78 cells were implanted into syngeneic mice. When tumors reached ˜300 mm³, 1×10⁷ OPT-LIS-IL-2 was injected IT and tissues were collected at the indicated timepoints. L. monocytogenes burdens were quantified in the tumor (FIGS. 3D-3E), spleen (FIG. 3F), and liver (FIG. 3G). When tumors were collected, half was directly homogenized and homogenate was tested for IL-2 quantification (FIG. 3H), and the other half was soaked in RPMI to allow IL-2 to passively diffuse from the tissue followed by IL-2 quantification from the tumor immersion (FIG. 3I). B78 cells were co-cultured with OPT-LIS-IL-2 (FIG. 3J) for 6 hours prior to standard DiffQuick staining for bacteria. N=3 mice/group.

FIGS. 4A-4F show that OPT-LIS-IL-2 persist in tumors and offers tumor control. 2×10⁶ B78 cells were implanted into the flank of syngeneic mice. When tumors reached ˜100 mm³, 1×10⁷ of the indicated strain was injected IT and tumors (FIGS. 4A-4B) were tracked for growth. At 35 days post treatment for OPT-LIS-IL-2 or LADD, tumors (FIG. 4C), spleens (FIG. 4D) and livers (FIG. 4E) were collected and quantified for L. monocytogenes burdens and tumor homogenates were quantified for IL-2 by ELISA (FIG. 4F). N=4 mice/group except where individual data points are shown. Significance was determined by one-way ANOVA of area under the curve of tumor growth curves. * p<0.05.

FIG. 5 shows the antitumor effect of OPT-IL-2 in combination with radiotherapy (RT) and anti-CTLA-4 (C4), with PBS and the LADD strain acting as controls. The top row shows mouse tumor volumes after receiving PBS, LADD, or OPT-LIS-IL-2. The second row shows mouse tumor volumes after receiving RT plus PBS, LADD, or OPT-LIS-IL-2. The third row shows mouse tumor volumes after receiving C4, RT and PBS, LADD, or OPT-LIS-IL-2, with the graph at the far right showing mouse tumor volume after receiving RT, anti-CTLA-4 and 14.18-IL2 immunocytokine (IC).

### DETAILED DESCRIPTION

It is to be appreciated that certain aspects, modes, embodiments, variations and features of the present technology are described below in various levels of detail in order to provide a substantial understanding of the present technology. The definitions of certain terms as used in this specification are provided below. Unless defined otherwise, all technical and scientific terms used herein generally have the same meaning as commonly understood by one of ordinary skill in the art to which this present technology belongs.

### I. Definitions

The following terms are used herein, the definitions of which are provided for guidance.

As used herein, the singular forms “a,” “an,” and “the” designate both the singular and the plural, unless expressly stated to designate the singular only.

The term “about” and the use of ranges in general, whether or not qualified by the term about, means that the number comprehended is not limited to the exact number set forth herein, and is intended to refer to ranges substantially within the quoted range while not departing from the scope of the present technology. As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein, “administration” of an agent, drug, bacterial strain(s), or composition of the present technology to a subject includes any route of introducing or delivering to a subject a compound to perform its intended function. Administration can be carried out by any suitable route, including intratumorally, orally, intranasally, parenterally (intravenously, intramuscularly, intraperitoneally, or subcutaneously), topically, or by inhalation. In some embodiments, the compositions are formulated for intravenous or intratumoral delivery. As used herein, administration includes self-administration and administration by another.

As used herein, “LADD” refers to a Live Attenuated Double Deleted L. monocytogenes strain known in the art. Attenuation of the strain is achieved by deletion of two virulence genes, actA and inlB, preventing cell-to-cell spread and hepatotoxicity, respectively.

As used herein, the terms “effective amount,” or “therapeutically effective amount,” and “pharmaceutically effective amount” refer to a quantity sufficient to achieve a desired therapeutic and/or prophylactic effect, e.g., an amount which results in the prevention of a disease, condition, and/or symptom(s) thereof. In the context of therapeutic or prophylactic applications, the amount of a composition administered to the subject will depend on the type and severity of the disease and on the characteristics of the subject, such as general health, age, sex, body weight, and tolerance to the composition drugs. It will also depend on the degree, severity, and type of disease or condition. The skilled artisan will be able to determine appropriate dosages depending on these and other factors. In some embodiments, multiple doses are administered. Additionally or alternatively, in some embodiments, multiple therapeutic compositions or compounds are used (e.g., pharmaceutical compositions comprising multiple bacterial strains alone or in combination with additional active agents, such as checkpoint blockade inhibitors or radiation therapies). In the methods described herein, compositions comprising the bacterial strains of the present technology may be administered to a subject having one or more signs, symptoms, or risk factors of cancer. For example, a “therapeutically effective amount” of the compositions of the present technology, includes levels at which the presence, frequency, or severity of one or more signs, symptoms, or risk factors of cancer are, at a minimum, ameliorated. In some embodiments, a therapeutically effective amount is achieved by multiple administrations. In some embodiments, a therapeutically effective amount is achieved with a single administration.

As used herein, “pharmaceutically acceptable carrier and/or diluent” or “pharmaceutically acceptable excipient” includes but is not limited to solvents, dispersion media, coatings, antibacterial and antifungal agents, isotonic and absorption delaying agents, and the like. In some embodiments, the pharmaceutically acceptable carrier comprises a polysaccharide, locust bean gum, an anionic polysaccharide, a starch, a protein, sodium ascorbate, glutathione, trehalose, sucrose, or pectin. In some embodiments, the polysaccharide comprises a plant, animal, algal, or microbial polysaccharide. In some embodiments, the polysaccharide comprises guar gum, inulin, amylose, chitosan, chondroitin sulphate, an alginate, or dextran. In some embodiments, the starch comprises rice starch. The use of such media and agents for biologically active substances is well known in the art. Further details of excipients are provided below. Supplementary active ingredients, such as antimicrobials, for example antifungal agents, can also be incorporated into the compositions.

As used herein, “pharmaceutically acceptable excipient” refers to substances and compositions that do not produce an adverse, allergic, or other untoward reaction when administered to an animal or a human. As used herein, the term includes all inert, non-toxic, liquid or solid fillers, or diluents that do not react with the therapeutic substance of the present technology in an inappropriate negative manner, solvents, dispersion media, coatings, antibacterial and antifungal agents, isotonic and absorption delaying agents, preservatives and the like, for example liquid pharmaceutical carriers e.g., sterile water, saline, sugar solutions, Tris buffer, ethanol and/or certain oils.

As used herein, “prevention,” “prevent,” or “preventing” of a disorder or condition refers to, in a statistical sample, reduction in the occurrence or recurrence of the disorder or condition in treated subjects/samples relative to an untreated controls, or refers delays the onset of one or more symptoms of the disorder or condition relative to the untreated controls.

As used herein “subject” and “patient” are used interchangeably. In some embodiments, the subject is an animal subject. In some embodiments, the animal subject is a mammal. In some embodiments, the mammalian subject is a human.

As used herein, the term “simultaneous” administration refers to the administration of at least two agents by the same route and at the same time or at substantially the same time.

As used herein, the term “separate” administration refers to an administration of at least two agents at the same time or at substantially the same time by different routes.

As used herein, the term “sequential” administration refers to administration of at least two agents at different times, the administration route being identical or different. More particularly, sequential use refers to the whole administration of one agent before administration of the other agent(s) commences. It is thus possible to administer one of the agents over several minutes, hours, or days before administering another.

A “synergistic therapeutic effect” refers to a greater-than-additive therapeutic effect which is produced by a combination of at least two therapeutic agents, and which exceeds that which would otherwise result from the individual administration of the agents. For example, use of bacterial strain(s) of the present technology in conjunction with other agents for the treatment of cancer may result in a greater than additive therapeutic effect. In some embodiments, the synergistic effect may permit the use of lower doses of bacterial strain(s) of the present technology and/or other agents than would be required if each were used alone.

“Treating,” “treat,” “treated,” or “treatment” of a disease or disorder includes: (i) inhibiting the disease or disorder, i.e., arresting its development; (ii) relieving the disease or disorder, i.e., causing its regression; (iii) slowing progression of the disorder; and/or (iv) inhibiting, relieving, or slowing progression of one or more symptoms of the disease or disorder.

It is to be appreciated that the various modes of treatment or prevention of medical diseases and conditions as described are intended to mean “substantial,” which includes total but also less than total treatment or prevention, and wherein some biologically or medically relevant result is achieved.

### II. Cancer Treatment and IL-2, IL-12, and CD40L

Harnessing the immune system to treat cancer has become commonplace due to the revolutionizing impact of immunotherapy¹. Despite these successes not all patients respond to immunotherapies and many eventually progress in their disease². Thus, more work is needed to develop broadly efficacious treatments that lead to durable responses and help fill the gap between responders and non-responders. CD8+ T-cell frequency is typically associated with greater overall survival³ and increased response to immunotherapy treatments such as checkpoint inhibitors (CPIs) 4. Yet for those with fewer CD8+ T-cells whose tumors are considered immunologically cold, the response to CPI treatments is less favorable⁴. Thus, treatments aimed at generating large numbers of highly functional, tumor-specific CD8+ T-cells and creating an immunologically hot tumor microenvironment to support those T-cells are needed to fill the gap.

IL-2 is a cytokine that stimulates the proliferation and survival of T-cells. High-dose recombinant IL-2 has been used as a treatment for renal cell carcinoma and metastatic melanoma with some success⁵. Despite some successes use of high-dose IL-2 has been limited due to severe treatment-related toxicities and a short in vivo half-life requiring many frequent administrations^{6,7}.

IL-12 is a cytokine that has a wide range of anti-tumor functions including activation of T-cells and NK cells to increase IFNγ production. Recombinant IL-12 has been demonstrated to potently induce tumor regression and has demonstrated synergy with IL-2 when delivered intratumorally⁴⁷⁻⁵⁰. Despite these promising results, the requirement for repeated intratumoral administration due to toxicity associated with systemic IL-12 delivery has limited further successful development of IL-12 in the clinic⁵¹.

CD40L is an activating ligand for CD40 which has many functions including promoting macrophage activation and antigen presentation as well as promoting dendritic cell maturation and antigen presentation among other functions⁵². Activation of CD40 signaling by either antibody mediated or CD40L mediated activation has demonstrated a synergistic effect in combination with radiotherapy and chemotherapy⁵³⁻⁵⁷. Similar to IL-2 and IL-12 mediated therapies, full development of anti-CD40 or CD40L therapeutics have been limited by toxicity associated with systemic delivery and signaling.

### III. Listeria monocytogenes Clinical Applications

Listeria monocytogenes is a gram-positive, facultative intracellular bacterium that has been employed in many clinical trials as an anti-cancer vaccine for numerous cancer types⁹⁻¹¹. The bacterium has been used for decades to understand aspects of immunology and cell biology¹². As a result, genetic tools have been developed that enable genetic engineering of the bacterium¹³. In clinical trials, L. monocytogenes has been engineered to express peptide fragment cancer antigens or larger proteins that are processed into antigenic fragments, and spurs a strong CD8+ T-cell response¹⁴. However, L. monocytogenes has not been shown to be an effective expression vector for full length, immune modulating proteins with secondary structures, and the cancer antigen expressing L. monocytogenes strains only produced cellular localized antigens.

Wild-type L. monocytogenes gains intracellular access through phagocytosis by professional phagocytes or by receptor mediated endocytosis mediated by two surface proteins, InlA and InlB into non-phagocytes^{9,15,16}. Once internalized L. monocytogenes is initially encased in a vacuole where it uses a cholesterol-dependent pore forming protein called Listerialysin O (LLO) to break free of the vacuole and into the cytosol¹⁷. The expression and function of LLO is tightly regulated as strains that fail to compartmentalize LLO function lyse any cell they infect¹⁸. Once in the cytosol, L. monocytogenes spreads cell to cell by hijacking the host actin cytoskeleton using the secreted L. monocytogenes protein, ActA^{19,20}.

Strains of L. monocytogenes used in clinical settings have been attenuated to be safe for application in human patients. The vaccine strain of L. monocytogenes used in this study, LADD (Live Attenuated Double Deleted), is attenuated by deletion of two virulence genes, actA and inlB, preventing cell to cell spread and hepatotoxicity, respectively¹⁰. This strain is 1,000-fold less virulent than wild-type L. monocytogenes yet retains its immunogenicity¹⁰. Importantly, LADD has previously been engineered to express and secrete both, exogenous antigens to drive antigen specific CD8+ T-cell responses, as well as mammalian proteins to modulate host cell physiology²¹⁻²⁵. During administration to a healthy mouse, LADD is taken into phagocytic cells of the liver and spleen where it establishes an intracellular niche and stimulates a robust CD8+ T-cell response^{19,26}. When applied to a tumor-bearing mouse, it was observed that L. monocytogenes is cleared or persists at very low levels in the spleen and liver yet accumulates in the tumor microenvironment (TME)^{27,28, and unpublished data}.

The present technology takes advantage of L. monocytogenes accumulation in the tumor microenvironment (TME) to deliver tumor-targeted immunomodulatory cytokines. Specifically, to express human IL-2 from L. monocytogenes as an in-situ vaccine to drive proliferation of tumor-specific T-cells. This disclosure demonstrates the production of bioactive IL-2 and accumulation of IL-2 engineered L. monocytogenes in the TME. Accordingly, the technology is useful for therapeutic methods for the treatment of cancer.

### IV. Therapeutic and Prophylactic Methods

The following discussion is presented by way of example only, and is not intended to be limiting.

One aspect of the present technology includes methods of treating or preventing cancer in a subject diagnosed as having, suspected as having, or at risk of having cancer. In therapeutic applications, compositions or medicaments comprising the L. monocytogenes bacterial strains of the present technology are administered to a subject suspected of, or already suffering from, cancer, in an amount sufficient to cure, or at least partially arrest, the signs or symptoms of cancer, including its complications and intermediate pathological phenotypes in development of the disease.

Subjects suffering from cancer can be identified by any or a combination of diagnostic or prognostic assays known in the art. For example, typical symptoms of cancer include, but are not limited to: tumor appearance, tumor growth, tumor metastasis, changes in weight, changes in skin coloration, sores that do not heal, changes in bowel or bladder habits, persistent cough, trouble breathing, difficult swallowing, hoarseness, indigestion or discomfort after eating, unexplained muscle pain, joint pain, fevers, night sweats, bleeding, or bruising, or any combination thereof. In some embodiments cancer is diagnosed using imaging technology, such as MRI, CT scans, ultrasounds, PET scans, and X-rays, for abnormalities. In some embodiments, cancer is diagnosed using genetic testing. In some embodiments, cancer is diagnosed by assaying tissue samples or biopsies for abnormalities.

In some embodiments, subjects with cancer treated with the bacterial strain(s) of the present technology, or spores thereof, will show amelioration or elimination of one or more of the following symptoms: tumor appearance, tumor growth, tumor metastasis, changes in weight, changes in skin coloration, sores that do not heal, changes in bowel or bladder habits, persistent cough, trouble breathing, difficult swallowing, hoarseness, indigestion or discomfort after eating, unexplained muscle pain, joint pain, fevers, night sweats, bleeding, or bruising, or any combination thereof.

### V. Modes of Administration and Effective Dosages

Compositions of the present technology for use in preventing, ameliorating, or treating cancer and/or reducing the severity of one or more risk factors, signs, or symptoms associated with cancer include genetically engineered L. monocytogenes bacterium, wherein the bacterium comprises a nucleic acid comprising a sequence encoding IL-2, IL-12, or CD-40L. The compositions of the present technology are administered to the subject in effective amounts (i.e., amounts that have desired therapeutic effect). The dose and dosage regimen will depend upon the nature of the cancer, the degree of cancer progression and symptom severity in the subject, the characteristics of the strain used, e.g., its therapeutic index, the subject, and the subject's history. The effective amount may be determined during pre-clinical trials and clinical trials by methods familiar to physicians and clinicians.

Additional components of the compositions of the present technology may include a preservative selected from the group consisting of sucrose, sodium ascorbate, and glutathione. In some embodiments the preservative is a cryoprotectant selected from the group consisting of a nucleotide, a disaccharide, a polyol, and a polysaccharide. In some embodiments, the cryoprotectant is selected from the group consisting of inosine-5′-monophosphate (IMP), guanosine-5′-monophosphate (GMP), adenosine-5′-monophosphate (AMP), uranosine-5′-monophosphate (UMP), cytidine-5′-monophosphate (CMP), adenine, guanine, uracil, cytosine, guanosine, uridine, cytidine, hypoxanthine, xanthine, orotidine, thymidine, inosine, trehalose, maltose, lactose, sucrose, sorbitol, mannitol, dextrin, inulin, sodium ascorbate, glutathione, and skim milk.

The genetically engineered L. monocytogenes bacterium, wherein the bacterium comprises a nucleic acid comprising a sequence encoding IL-2, IL-12, or CD-40L, described herein can be incorporated into pharmaceutical compositions for administration, singly or in combination, and given to a subject for the treatment or prevention of a disorder described herein. Such compositions typically include the active agent and a pharmaceutically acceptable carrier. As used herein the term “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions. Carriers can be solid-based dry materials for formulations in powdered form, and can be liquid or gel-based materials for formulations in liquid or gel forms, which forms depend, in part, upon the routes or modes of administration. In some embodiments, the pharmaceutically acceptable carrier comprises a polysaccharide, locust bean gum, an anionic polysaccharide, a starch, a protein, sodium ascorbate, glutathione, trehalose, sucrose, or pectin. In some embodiments, the polysaccharide comprises a plant, animal, algal, or microbial polysaccharide. In some embodiments, the polysaccharide comprises guar gum, inulin, amylose, chitosan, chondroitin sulphate, an alginate, or dextran. In some embodiments, the starch comprises rice starch.

Pharmaceutical compositions are typically formulated to be compatible with the intended route of administration. Examples of routes of administration include intratumorally, orally, intranasally, parenterally (intravenously, intramuscularly, intraperitoneally, or subcutaneously), topically, or by inhalation. In some embodiments, the compositions of the present technology are formulated for intravenous administration. In some embodiments, the compositions of the present technology are formulated for intratumoral administration. Other formulations will be readily apparent to one skilled in the art.

Dosage, toxicity and therapeutic efficacy of any therapeutic agent can be determined by standard pharmaceutical procedures in cell cultures or experimental animals. The data obtained from the cell culture assays and animal studies can be used in formulating a range of dosage for use in humans. The dosage of such compounds may be within a range of circulating concentrations that include the ED50 with little or no toxicity. The dosage may vary within this range depending upon the dosage form employed and the route of administration utilized. For any compound used in the methods, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to determine useful doses in humans accurately.

In some embodiments, the compositions of the present technology comprise one or more genetically engineered L. monocytogenes strains comprising a nucleic acid comprising a sequence encoding IL-2, IL-12, or CD-40L, ranging from at least about 10⁶ colony forming units (CFU)/mL to at least about 10⁹ CFU/mL, or any value in between. In some embodiments, the compositions range from about 10⁶ CFU/mL to about 10⁹ CFU/mL. In some embodiments, the compositions range from about 10⁷ CFU/mL to about 10⁹ CFU/mL. In some embodiments, the compositions range from about 10⁸ CFU/mL to about 10⁹ CFU/mL.

An exemplary treatment regimen entails administration of at least a single dose of the one or more genetically engineered L. monocytogenes strains of the present technology to a subject. In some embodiments, the subject receives a single dose of the one or more genetically engineered L. monocytogenes strains. In some embodiments, the subject receives multiple doses of the one or more genetically engineered L. monocytogenes strains. In some embodiments, the multiple doses are administered about 1 day apart, about 2 days apart, about 3 days apart, about 4 days apart, about 5 days apart, about 6 days apart, about 1 week apart, about 2 weeks apart, about 3 weeks apart, about 4 weeks apart, about two months apart, about 3 months apart, about 4 months apart, about 5 months apart, about 6 months apart, about 7 months apart, about 8 months apart, about 9 months apart, about 10 months apart, about 1 year apart, or about 2 years apart. In some embodiments the multiple doses are administered at regular or irregular intervals. In some embodiments, the subject receives a single dose of the one or more genetically engineered L. monocytogenes strains of the present technology, and receives a second dose once the one or more genetically engineered L. monocytogenes strains are no longer detected in one or more tumors in the subject. In therapeutic applications, a relatively high dosage at relatively short intervals is sometimes required until progression of the disease is reduced or terminated, or until the subject shows partial or complete amelioration of symptoms of disease. Thereafter, the subject can be administered a prophylactic regime. In some embodiments, compositions of the present technology are administered multiple times per day. In some embodiments, compositions of the present technology are administered to a subject once, twice, or three times per day or more for a certain period of time or until the subject is deemed cured of primary disease, not to be at risk for recurrence of primary disease, or not to be at risk for the disease. In some embodiments, the compositions of the present technology may be administered to the subject for the remainder of the subject's life.

In some embodiments, administration is paired with an exposure to co-therapeutics (i.e., agents known in the art for the treatment of cancer, such as immune checkpoint blockade inhibitors or radiation therapies), either simultaneously, separately, or sequentially with dosing of the compositions of the present technology. In some embodiments, administration of radiation therapy is combined simultaneously, separately, or sequentially with dosing of the compositions of the present technology. In some embodiments, administration of immune checkpoint blockade inhibitor therapy is combined simultaneously, separately, or sequentially with dosing of the compositions of the present technology. In some embodiments, administration of radiation therapy and immune checkpoint blockade inhibitor therapy are combined simultaneously, separately, or sequentially with dosing of the compositions of the present technology. In some embodiments, compositions of the present technology, radiation therapy, and immune checkpoint blockade inhibitor therapy are administered simultaneously, separately, or sequentially to a subject. In any of the preceding embodiments, the one or more co-therapeutics and the compositions of the present technology can be administered by any appropriate route and can be formulated for any appropriate route of administration. For example, in some embodiments the one or more genetically engineered L. monocytogenes strains, the radiation therapy, and the immune checkpoint blockade therapy are formulated for intratumoral, intraperitoneally, or intravenous administration. In some embodiments, the one or more genetically engineered L. monocytogenes strains, the radiation therapy, and the immune checkpoint blockade therapy are administered intratumorally, intraperitoneally, or intravenously. In some embodiments, the radiation therapy is administered via an external beam. In some embodiments, any of the combination treatments described herein result in a synergistic therapeutic effect. For example, administration of compositions of the present technology with one or more additional therapeutic agents for the prevention or treatment of cancer will have greater than additive effects in the prevention or treatment of the disease and/or one or more of its signs or symptoms.

In any case, the multiple therapeutic agents may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. In addition, the combination methods, compositions and formulations are not to be limited to the use of only two agents.

The skilled artisan will appreciate that certain factors may influence the dosage and timing required to effectively treat a subject, including but not limited to, the severity of the disease or disorder, previous treatments, the general health and/or age of the subject, and other diseases present. Moreover, treatment of a subject with a therapeutically effective amount of the therapeutic compositions described herein can include a single treatment or a series of treatments.

### EXAMPLES

### Example 1: Materials and Methods

This example describes materials and methods used to generate and test L. monocytogenes strains described herein.

### Bacterial Strains and Construction

E. coli was regularly grown at 37° C. in LB or on LB+1.5% agar plates and frozen in LB+40% glycerol. XL-1 Blue E. coli was used for subcloning and propagation of plasmids and S17 E. coli was used for conjugation into L. monocytogenes. L. monocytogenes were routinely grown at 37° C. in brain-heart infusion (BHI, [BD, 237500]) or on BHI+1.5% agar plates and frozen in BHI+40% glycerol. Antibiotics were used at the following concentrations: 200 μg/ml streptomycin or 30 μg/ml kanamycin.

Plasmids pIMK2-ActAN30-IL2 and pIMK2-ActAN100-IL2 were constructed by Gibson Assembly (New England Biolabs, E2621) using NcoI-HF (New England Biolabs, R3193) and SalI-HF (New England Biolabs, R3138) linearized pIMK2 and a gBlock (IDT) comprised of the ActA N30 or N100 amino acids fused to human IL2 (sequences in Table 1). Plasmid pIMK2-hly5′UTR-ActAN100-IL2 was constructed by Gibson Assembly using NcoI-HF linearized and Quick CIP (New England Biolabs, M0525) treated pIMK2-ActAN100-IL2, and a 46 base-pair fragment of the hly 5′ UTR amplified from genomic DNA by primers phelp-hly5′ utr_fwd and phelp-hly5′ utr_rev (sequence in Table 1). Plasmids were cloned first into XL1-Blue E. coli and selected for on kanamycin plates and sequence confirmed by full-plasmid sequencing (plasmidsaurus) and then subcloned into S17 E. coli for subsequent conjugation. Conjugations were performed by spreading a single S17 colony onto a BHI-agar plate in a 1 cm square, followed by spreading of a corresponding L. monocytogenes colony in perpendicular fashion over the first colony. This was grown overnight at 37° C., and the patch was then scraped into 400 μL PBS, and diluted in 10-fold serial dilutions and plated onto antibiotic-containing plates to select against S17 and for plasmid integration into L. monocytogenes. Single colonies were screened by PCR for the hly gene (hly-F, and hly-R) and for the IL-2 construct. IL-2 production was then confirmed by ELISA.

All strains of L. monocytogenes used in this study were in the 10403S background. The attenuated strain used in this study, termed Live Attenuated Double Deleted (LADD) is genetically modified by in-frame deletion of the virulence factors, actA and inlB (ΔactA and ΔinlB) and is previously described¹⁰. Strain LIS-IL-2N100 (referred to as LIS-IL-2) was constructed by conjugating pIMK2-ActAN100-IL-2 into LADD. The strain LIS-IL-2N30 was constructed by conjugating pIMK2-ActAN30-IL-2 into LADD. The strain OPT-LIS-IL-2 was constructed by conjugating pIMK2-hly5′UTR-ActAN100-IL-2 into LADD.

### Cell Lines and Culture

B78 cells were routinely cultured in RPMI (Fisher, #11875093), with 10% FBS (Fisher, SH30071), 1% L-Glutamine (Fisher, #25030081), and 1% Antibiotic/Antimycotic (Fisher, 15240062). For infection experiments, Antibiotic/Antimycotic was omitted. MC38 and CT26 cells were routinely cultured in DMEM (Invitrogen, 11965092) with 10% FBS, 1% L-Glutamine, and 1% Antibiotic/Antimycotic. For infection experiments, 5×10⁵ B78 cells were plated in 24 well plates (Fisher, 12556006) onto flamed glass coverslips (VWR, 89015-725). Cells were then infected with indicated strains washed with PBS and grown overnight at 30° C. without agitation at an MOI=1. Infection proceeded until indicated timepoints, and media was filtered and stored at −80° C. until use, and coverslips were subjected to standard Diff-Quick staining protocols. Coverslips were then mounted and imaged under a brightfield microscope using a 50× objective.

CTLL-2 cells were maintained in RPMI+10% FBS, 1% L-Glutamine+200 U/mL IL-2. Cells were washed to remove residual IL-2 and 5×10³ cells were added to 96 well plates and media was mixed ½ with 0.22 μM filtered RPMI media inoculated with 1×10⁸ IL-2 secreting L. monocytogenes and grown for 1 hour at 37° C. with agitation. Cells were cultured for 24 hours in the presence of [³H]thymidine and incorporation was measured by liquid scintillation counting.

### Tumor Experiments and Treatments

2×10⁶ B78, or MC38 cells were injected intradermally into the right flank of syngeneic C57BL/6 mice (Taconic Farms) or 2×10⁶ CT26 were injected intradermally into the right flank of syngeneic BALB/C mice (Taconic Farms). Tumors were measured at the indicated timepoints using electronic calipers and tumor volume was calculated according to tumor volume=0.5× ((small width) 2×(large width)) where the large width was the widest point in the tumor, and the small width was the widest corresponding perpendicular width. When tumors reached the indicated size or timepoint, treatment with L. monocytogenes was performed by injection of 1×10⁷ bacteria resuspended in PBS in 50 μL directly into the tumor using a 27 g needle, or by injection of 1×10⁷ bacteria resuspended in 200 μL PBS via the lateral tail vein using a 27 g needle. Tumor volumes were measured at the indicated timepoints until collection.

### Enumeration of Bacterial Burdens in Tissues and Collection

At the indicated timepoints, mice were euthanized, and tumors were collected into cold, sterile PBS, while livers (including the gallbladder) and spleens were collected into cold, sterile PBS+0.1% IPEGAL (Sigma, I8896-50ML). Tissues were then homogenized using a tissue grinder (Polytron, Pt-3100). In some experiments, tumors were cut in half, weighed, and ½ was subjected to homogenization in PBS immediately. The other half was soaked in 500 μL RPMI at 4° C. for 18 hours to allow cytokines to passively diffuse from the tissue and avoid cellular lysis from homogenization. Homogenates were then plated on BHI+agar+streptomycin plates and stored at 4° C. and CFU were enumerated the following day. CFU/tumor was determined by adjusting for percents of tumors homogenized versus used for soaking. Blood was collected by retroorbital bleed using heparinized capillary tubes (Fisher, 22260950) into serum separator tubes (Fisher, 365956). After a minimum of 10 minutes of coagulation at room temperature, separator tubes were spun at 3000×G for 10 minutes and cell free serum was collected into new tubes and plated for bacterial enumeration and frozen at −80° C. until use in ELISA.

### ELISAs and LegendPlex Assays

Medias, tissue homogenates, or mouse serum were clarified by spinning at max speed and then subjected to filtration through Nalgene sterile 25 mm PES 0.2 um syringe filters (ThermoFisher, 725-2520). Human IL-2 ELISA was performed using ELISA MAX™ Standard Set Human IL-2 (Biolegend, 431801) according to manufactures instructions. LEGENDplex™ Mouse B Effector ½ (Be1/2) Panel (8-plex) w/VbP (Biolegend, 740821) was performed according to manufacturer's instructions.

### ELISAs and LegendPlex Assays

Primers and other oligonucleotides used in these experiments are shown below in Table 1.

TABLE 1
gBlock and primer sequences used in this study

| ActAN30- | aggagagtgaaacccatggGtgggattaaatagatttatgcgtgcgatgatggtagttttcattactgc | SEQ ID NO: |
|---|---|---|
| IL2 | caactgcattacgattaaccccgacataatatttgcagcgCCAACATCTTCTTCCACTAAGAAGAC | 1 |
| | TCAGCTACAACTTGAACACCTTCTGTTGGACCTACAAATGATTCTA | |
| | AATGGAATAAACAATTATAAAAATCCTAAATTAACTAGAATGCTCA | |
| | CGTTCAAATTCTACATGCCGAAAAAAGCAACCGAGTTAAAACATTT | |
| | ACAATGTCTTGAAGAAGAACTTAAACCATTGGAAGAAGTGCTTAA | |
| | CTTGGCCCAAAGTAAAAATTTTCATTTACGTCCACGAGATTTAATT | |
| | AGCAATATCAATGTCATTGTATTAGAATTAAAAGGTAGTGAAACCA | |
| | CATTTATGTGCGAATATGCTGATGAGACAGCGACAATTGTTGAATT | |
| | TTTAAACCGCTGGATTACATTTTGTCAATCAATTATCTCGACGTTAA | |
| | CGTGAGTCGACCTCGAGGGG | |
| ActAN100- | gaaggagagtgaaacccatggGtgggattaaatagatttatgcgtgcgatgatggtagttttcattact | SEQ ID NO: |
| IL2 | gccaactgcattacgattaaccccgacataatatttgcagcgacagatagcgaagattccagtctaaa | 2 |
| | cacagatgaatgggaagaagaaaaaacagaagagcagccaagcgaggtaaatacgggaccaagatacga | |
| | aactgcacgtgaagtaagttcacgtgatattgaggaactagaaaaatcgaataaagtgaaaaatacgaa | |
| | caaagcagacctaatagcaatgttgaaagcaaaagcagagaaaggtggatccCCAACATCTTCTTCCAC | |
| | TAAGAAGACTCAGCTACAACTTGAACACCTTCTGTTGGACCTACAAATGATTCTA | |
| | AATGGAATAAACAATTATAAAAATCCTAAATTAACTAGAATGCTCA | |
| | CGTTCAAATTCTACATGCCGAAAAAAGCAACCGAGTTAAAACATTT | |
| | ACAATGTCTTGAAGAAGAACTTAAACCATTGGAAGAAGTGCTTAA | |
| | CTTGGCCCAAAGTAAAAATTTTCATTTACGTCCACGAGATTTAATT | |
| | AGCAATATCAATGTCATTGTATTAGAATTAAAAGGTAGTGAAACCA | |
| | CATTTATGTGCGAATATGCTGATGAGACAGCGACAATTGTTGAATT | |
| | TTTAAACCGCTGGATTACATTTTGTCAATCAATTATCTCGACGTTAA | |
| | CGTGAGTCGACCTCGAGGGGGGG | |
| phelp- | agggaacaaaagctgggtacCCATTATGCTTTGGCAGTTTATTC | SEQ ID NO: |
| hly5′ | | 3 |
| utr fwd | | |
| phelp- | agcctgacatGGGTTTCACTCTCCTTCTAC | SEQ ID NO: |
| hly5′ | | 4 |
| utr rev | | |
| hly-F | TCAACCAGATGTTCTCCCTGTA | SEQ ID NO: |
| | | 5 |
| hly-R | CACTGTAAGCCATTTCGTCATC | SEQ ID NO: |
| | | 6 |

### Statistics and Analysis

Statistical analysis was performed by GraphPad Prism Software (La Jolla, CA) and analyzed via Kruskal-Wallace, Mann Whitney or one-way ANOVA with Tukey's correction as indicated. For tumor experiments, Prism was used to calculate the area under the curve at the last timepoint with all mice surviving and one-way ANOVA with Tukey's correction was used to determine significance.

### Example 2: Construction and Characterization of L. monocytogenes Expressing IL-2

This example demonstrates the engineering of L. monocytogenes strain for the production of biologically active IL-2.

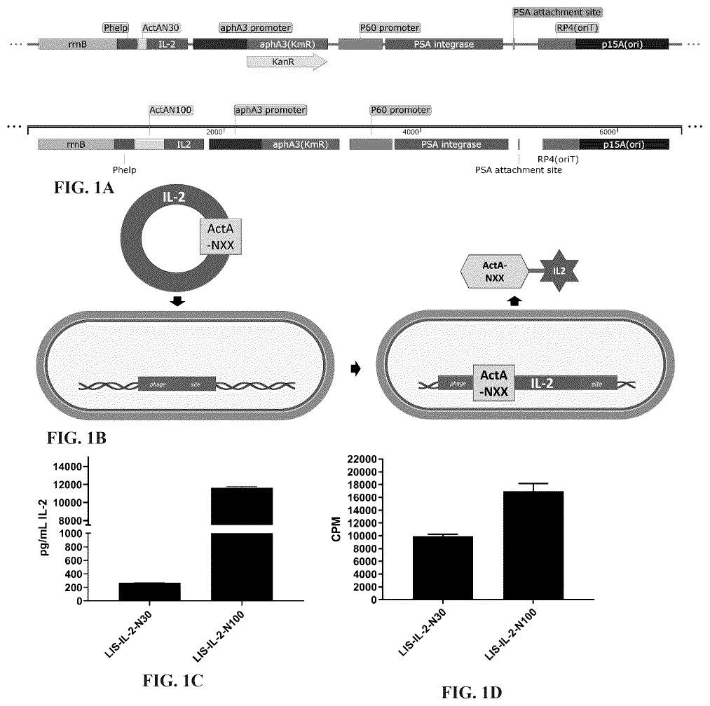

To create L. monocytogenes capable of secreting human IL-2, a Listeria-codon optimized sequence of the human interleukin-2 (IL-2) gene was fused to the n-terminal 30 or 100 amino acids (termed N30 or N100) of the L. monocytogenes secreted protein ActA as previously described^{21,36} Fusion to ActA N30 or N100 facilitates secretion from the bacterium; the N30 fusion results in an untagged IL-2 while N100 results in 70 amino acids of ActA remaining on the secreted IL-2. These constructs were cloned into an L. monocytogenes expression vector (pIMK2) that integrates stably into the bacterium's genome at the attBB′ site in the tRNA^{Arg} locus^{37, 46}, generating pIMK2-ActAN100-IL-2 and pIMK2-ActAN30-IL-2 which were then conjugated into the attenuated L. monocytogenes background, LADD (FIGS. 1A-1B).

To determine whether the engineered L. monocytogenes strains were capable of secreting IL-2, IL-2 production was assessed by ELISA from L. monocytogenes grown in RPMI media. L. monocytogenes carrying the ActA N30 and ActA N100 constructs secreted IL-2, however the N100 variant produced more IL-2 than the N30 variant (FIG. 1C). To test the hypothesis that IL-2 secreted by L. monocytogenes retained biological activity, proliferation of IL-2 responsive CTLL-2 cells was assessed in response to supernatants from IL-2-secreting L. monocytogenes in the presence of [³H]thymidine for 24 hours. Measuring [³H]thymidine incorporation by liquid scintillation counting, it was found that both L. monocytogenes strains engineered to secrete IL-2 induced CTLL-2 proliferation and, consistent with the ELISA data, L. monocytogenes harboring the N100-IL-2 fusion induced greater [³H]thymidine incorporation than the N30-IL-2 fusion (FIG. 1D). Taken together, these data demonstrate that the L. monocytogenes constructs express and secrete functional IL-2 that retains functionality on mammalian cells expressing the cognate receptors. Based on the increased production of IL-2 by the N100 variant, combined with the retained biological activity of IL-2, the N100 variant, hereafter called LIS-IL-2, was used for subsequent experiments.

These results demonstrate that engineered L. monocytogenes of the present disclosure is effective for the production of biologically active IL-2. Accordingly, it is effective in therapeutic methods comprising the production of IL-2, such as in the treatment of cancer.

### Example 3: Engineered L. monocytogenes Accumulates and Produces IL-2 in Tumor Microenvironments

This example demonstrates that engineered L. monocytogenes of the present disclosure accumulates and produces IL-2 in tumor microenvironments.

It was then determined whether LIS-IL-2 would accumulate in the tumors of B78-tumor bearing mice and secrete IL-2, while being cleared from normal L. monocytogenes target tissues. To test this hypothesis, B78 tumors were implanted into the flank of syngeneic mice, and when the tumors reached 300 mm³ on average, mice were treated with 1×10⁷LIS-IL-2 or LADD (which does not secrete IL-2) as a control by intratumoral (IT) or intravenous (IV) injection. At 1-, 3- and 7-days post treatment serum, livers, spleens, and tumor tissue were collected and assessed for L. monocytogenes burdens.

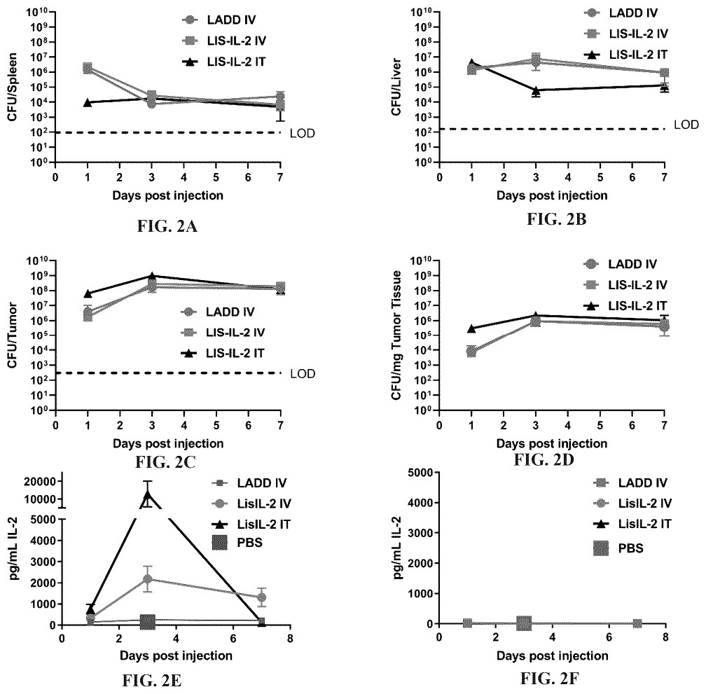

L. monocytogenes was detected in all tissues (FIGS. 2A-C) of immunized mice, but not in the serum (data not shown), suggesting that the bacteria is rapidly cleared from circulation. Burdens decreased over time in the spleen following IV administration but remained constant following IT administration (FIG. 2A). Splenic burdens were equivalent by day 7 post treatment for both treatments. Liver burdens were slightly higher than in the spleen. When treatment was applied IT, a decreased burden in the liver compared to IV was observed, however, as in the spleen, burdens eventually normalized (FIG. 2B). There were no apparent differences between LADD and LIS-IL-2 L. monocytogenes tissue clearance. In contrast to the peripheral tissues, tumor burdens increased over time. When given IV, ˜1×10⁶ LIS-IL-2 or LADD were detected in tumors 24 hours post-treatment. IT administration yielded nearly 1×10⁸ bacteria at this early timepoint. This number increased for both LADD and LIS-IL-2, reaching a maximum of nearly 1×10⁹ bacteria per tumor (FIG. 2C). It is possible that there is a maximum carrying capacity of bacteria that can be supported by a given mass of tumor, as burdens maxed out at 1×10⁶ bacteria/mg of tumor tissue (FIG. 2D). These data demonstrates that attenuated L. monocytogenes persists in the spleen and liver in tumor-bearing mice in contrast to administration of the LADD strain to naïve mice where the strains are fully cleared within 72 hours of immunization¹⁰. It is possible that this is due to the accumulation of bacteria in the immunosuppressed TME which results in recurrent seeding of peripheral organs. Nevertheless, LADD-based vaccines have been utilized in various clinical trials at doses of 1×10⁹ bacteria/infusion in humans¹⁴, demonstrating the safety of this approach.

IL-2 levels were assessed in the tumors of immunized mice using an ELISA for human IL-2. IL-2 levels were below detection limits in the tumor of all mice treated with LADD, or PBS as a control. In contrast, LIS-IL-2 treatment yielded peak IL-2 concentrations in the tumor at 3-days post treatment, which appeared to decline by day 7. IT administration yielded a greater peak of almost 10 ng IL-2/tumor compared to 3 ng IL-2/tumor for IV administration (FIG. 2E). IL-2 was below the detection limit in all serum samples, suggesting that the LIS-IL-2 delivered IL-2 remains largely localized to sites of high bacterial burden (FIG. 2F). No overt signs of distress or illnesses were observed in treated mice compared to PBS controls. Collectively, this data demonstrates that L. monocytogenes accumulates in B78 tumors while being controlled in healthy tissue and demonstrates that L. monocytogenes can deliver cytokines specifically to the TME.

These results demonstrate that engineered L. monocytogenes strains of the present disclosure accumulate and produce IL-2 in tumor microenvironments. Accordingly, the strains effective in therapeutic methods comprising the production of IL-2, such as in the treatment of cancer.

### Example 4: Use of L. monocytogenes Expressing IL-2 for the Suppression of Tumor Growth

This example demonstrates the use of L. monocytogenes expressing IL-2 to slow tumor growth.

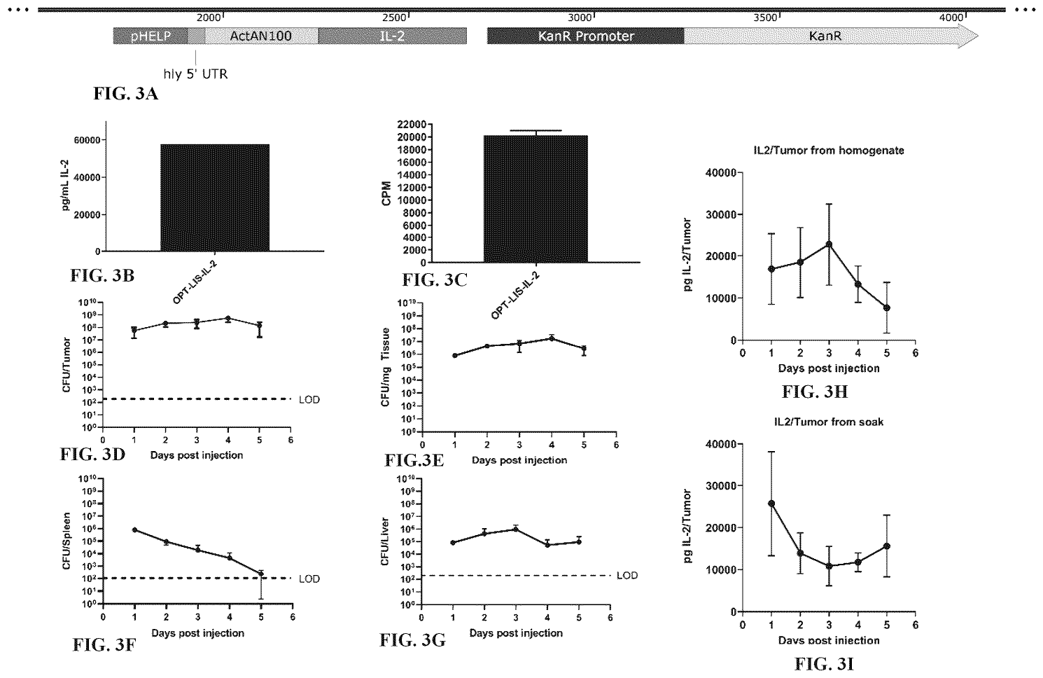

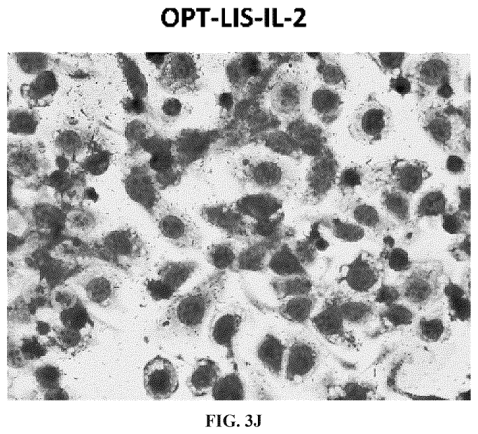

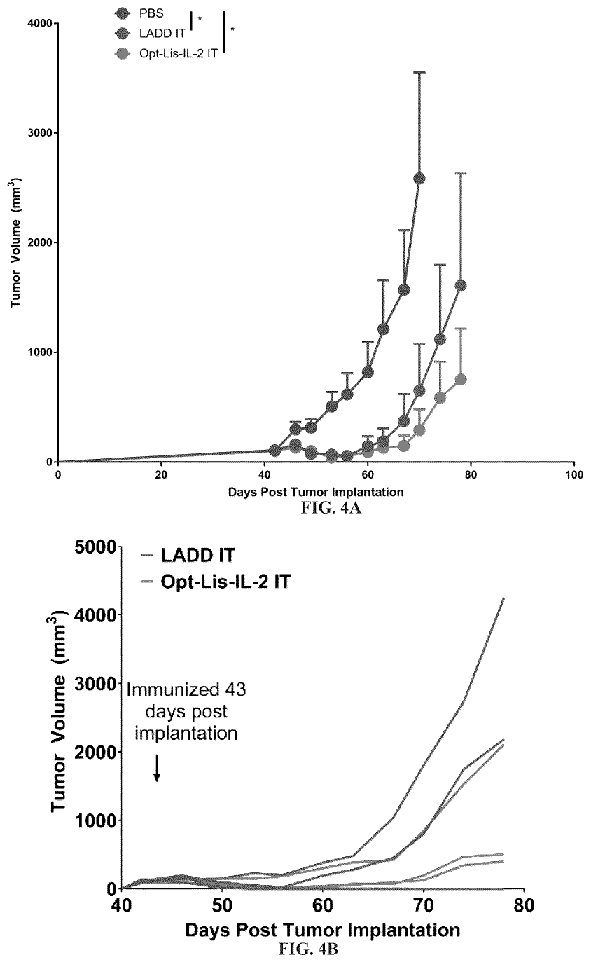

A second version of LIS-IL-2 with increased IL-2 expression was created named operational plasmid transformant LIS-IL-2 (OPT-LIS-IL-2). An expression construct was generated like that for LIS-IL-2 but with the 5′-UTR of the L. monocytogenes hly gene upstream of the ActAN100-IL-2 fusion construct (FIG. 3A). This UTR addition enhances protein expression, increasing translation in the bacterium³⁸. This construct was conjugated into LADD to generate OPT-LIS-IL-2. Increased IL-2 production was confirmed (˜60,000 μg/mL for OPT-LIS-IL-2 compared to ˜12,000 μg/mL for LIS-IL-2) and increased CTLL-2 proliferation (˜20,000 μg/mL for OPT-LIS-IL-2 compared to ˜18,000 μg/mL for LIS-IL-2) in supernatants from this strain compared to LIS-IL-2 (FIGS. 1C, 1D, 3B, 3C). OPT-LIS-IL-2 accumulates in tumor tissues reaching a max of ˜1×10⁹ bacteria/tumor (FIGS. 3D-3E). The new strain is cleared from the liver and spleen with comparable kinetics to LIS-IL-2 (FIGS. 3F-3G). OPT-LIS-IL-2 was cultured with B78 cells for 6 hours and cells had a normal appearance with what appear to be intracellular bacteria (FIG. 3J).

To address whether IL-2 produced by OPT-LIS-IL-2 was retained intracellularly, tumors from one through five days post treatment were collected and half the tumor was soaked in media for 18 hours to allow passive diffusion of cytokines from the tumor, while the other half was directly homogenized. Relatively similar amounts of IL-2 were detected in samples of OPT-LIS-IL-2 treated tumors that were directly homogenized compared to soaked (FIGS. 3H, 3I). When tumor samples were soaked, an initial peak was observed at one day post treatment, which decreased until day four and began rising again at day five post treatment (FIG. 3I). Overall, the levels of IL-2 detected in these samples was comparable to that of directly homogenized samples, indicating that IL-2 is predominantly extracellular.

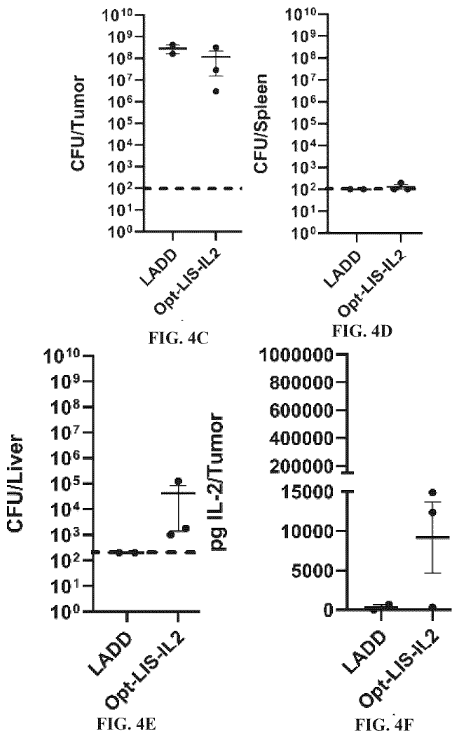

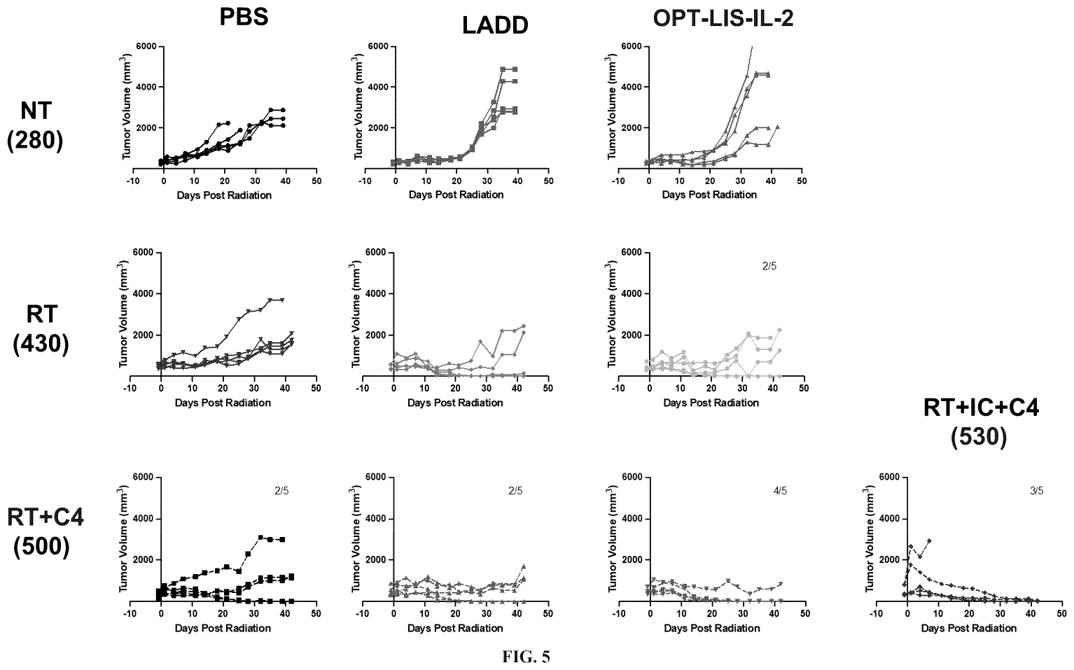

It was hypothesized that enhanced IL-2 expression in OPT-LIS-IL-2 would result in better tumor control relative to LADD. To test this hypothesis, B78 tumors were implanted into the flank of syngeneic B6 mice, and 1×10⁷ LADD, OPT-LIS-IL-2, or PBS was administered via IT injection when tumors reached ˜100 mm³. OPT-LIS-IL-2 treatment conferred enhanced tumor control compared to LADD, with diminished tumor volumes at multiple time points during the experiment (FIG. 4A). Of four mice treated with LADD, two completely cleared the B78 tumor without recurrence, whereas only one of four OPT-LIS-IL-2 treated mice completely cleared the tumor (FIG. 4B). Tumor shrinkage was observed for all mice treated with these two strains, and most tumors were nearly undetectable by day 10 post treatment. Of the mice that did not completely clear the tumors, those treated with LADD had more rapid tumor growth than the mice treated with OPT-LIS-IL-2 (FIG. 4B), which may suggest that when tumors are not completely cleared, OPT-LIS-IL-2 may control regrowth better than LADD.

To assess bacterial burdens and IL-2 production, the tumors, livers, and spleens of LADD and OPT-LIS-IL-2 were collected at day thirty-five post treatment due to differences in tumor growth rates. All strains tested persisted in B78 tumors at high burdens for many weeks after initial treatment (FIG. 4C). At these later timepoints, L. monocytogenes was nearly undetectable in the spleens (FIG. 4D) and only OPT-LIS-IL-2 treated mice had low but detectible burdens in the liver (FIG. 4E). At thirty-five days post treatment, there was an average of ˜10 ng IL-2/tumor in the OPT-LIS-IL-2-treated tumor homogenates, whereas none was detected in LADD-treated mice (FIG. 4F) suggesting that unlike the original LIS-IL-2 strains, IL-2 production in the TME persists with the OPT-LIS-IL2 strains. Taken together, these data demonstrate that the increased and persistent levels of IL-2 produced in the TME by OPT-LIS-IL-2 provide benefits to tumor control.

These results demonstrate that the delivery of IL-2 to tumor microenvironments by engineered L. monocytogenes of the present disclosure slows tumor growth. Accordingly, the engineered L. monocytogenes of the present disclosure are effective in therapeutic methods for the treatment of cancer.

### Example 5: Synergistic Benefits from OPT-LIS-IL-2 Co-Therapies

This example demonstrates the use of engineered L. monocytogenes of the present disclosure in combination with standard cancer therapies. Briefly, 2×10⁶ B78 tumor cells were injected on the right flank of mice. Mice were randomized 43 days later. The tumors were treated with either OPT-LIS-IL-2, OPT-LIS-IL-2 and radiation therapy (RT), or OPT-LIS-IL-2, RT, and anti-CTLA-4 (C4). The LADD strain and PBS were used as controls for the OPT-LIS-IL-2 combination therapies. The tumors were injected intratumorally with 0.1 ml of PBS, 1×10⁷ OPT-LIS-IL-2, or 1×10⁷ LADD. Tumors of RT treated mice were given 12 Gy of external beam radiation therapy (RT). C4 treated mice were injected intraperitoneally with 0.2 mg anti-CTLA-4 antibody (C4). Tumor growth was measured over time via calipers and tumor volume was estimated. FIG. 5 shows individual tumor volume over time, including noting how many of the tumors were rejected or cleared. Tumors receiving OPT-LIS-IL-2, RT, and anti-CTLA-4 had the greatest proportion of mice reject or clear their tumors, even in comparison with the standard treatment of RT, anti-CTLA-4 and 14.18-IL2 immunocytokine (IC) (RT+IC+C4). This experiment was repeated and showed similar results. In addition, B78 tumor-bearing mice treated with OPT-LIS-IL-2, RT, and anti-CTLA-4 had statistically significant longer survival (p=0.038) than mice treated with LADD, RT, and anti-CTLA-4 (data not shown).

### Example 6: L. monocytogenes IL-12 Expression Strain

This example will demonstrate the engineering of L. monocytogenes strain for the production of biologically active IL-12.

An L. monocytogenes IL-12 expression strain (Lis-IL-12) will be generated and testing for therapeutic efficacy against cancer. IL-12 is a dimer of IL-12p35 and IL-12p40. Briefly, Lis-IL-12 will be generated by operably linking an IL-12p35 (NCBI Gene ID: 16159) encoding transcript and an IL-12p40 (NCBI Gene Id: 16160) encoding transcript to L. monocyotgenes secretion signals, such as the ActAN secretion signal. In some embodiments, the IL-12 encoding transcripts encodes full-length IL-12p35 and IL-12p40. In some embodiments, the IL-12p35 encoding transcript and the IL-12p40 transcript encode a portion of IL-12p35 and IL-12p40 respectively. The IL-12 encoding transcripts may further comprise one or more untranslated regions that promote IL-12 expression, such as the hly 5′ UTR. The IL-12 encoding transcripts will then be incorporated into a vector for expression in L. monocytogenes, such as pIMK2, and a L. monocytogenes strain (e.g., LADD) will be transformed with said vector. The resulting Lis-IL-12 strain will then be assayed for release of biologically active IL-12 and therapeutic efficacy against one or more cancer models, optionally including the B78 tumor mouse models as described herein.

It is expected that these results will show that a Lis-IL-12 strain has been developed that releases biologically active IL-12 in vivo and that Lis-IL-12 is effective for treating or ameliorating one or more signs and symptoms of cancer. Accordingly, these results will show that Lis-IL-12 is effective in compositions and methods for the treatment of cancer.

### Example 7: L. monocytogenes CD40L Expression Strain

This example will demonstrate the engineering of L. monocytogenes strain for the production of biologically active CD40L.

An L. monocytogenes CD40L expression strain (Lis-CD40L) will be generated and testing for therapeutic efficacy against cancer. Briefly, Lis-CD40L will be generated by operably linking a CD40L (NCBI Gene ID: 21947) encoding transcript to an L. monocyotgenes secretion signal, such as the ActAN secretion signal. In some embodiments, the CD40L encoding transcript encodes full-length CD40L. In some embodiments, the CD40L encoding transcript encodes a portion of IL-12. The CD40L encoding transcript may further comprise one or more untranslated regions that promote CD40L expression, such as the hly 5′ UTR. The CD40L encoding transcript will then be incorporated into a vector for expression in L. monocytogenes, such as pIMK2, and a L. monocytogenes strain (e.g., LADD) will be transformed with said vector. The resulting Lis-CD40L strain will then be assayed for release of biologically active CD40L and therapeutic efficacy against one or more cancer models, optionally including the B78 tumor mouse models as described herein.

It is expected that these results will show that a Lis-CD40L strain has been developed that releases biologically active CD40L in vivo and that Lis-CD40L is effective for treating or ameliorating one or more signs and symptoms of cancer. Accordingly, these results will show that Lis-CD40L is effective in compositions and methods for the treatment of cancer.

### Example 8: L. monocytogenes IL-2/IL-12/CD40L Expression Strains for Treating Cancer in Human Subjects

This example will demonstrate the efficacy of engineered L. monocytogenes strains of the present technology for treating cancer in human subjects.

The one or more engineered strains of the present technology will be assayed for their therapeutic potential in treating cancer in humans. Briefly, one or more subjects diagnosed with cancer will be administered one or more of the engineered L. monocytogenes strains of the present technology (e.g., OPT-Lis-IL-2, Lis-IL-12, Lis-CD40L). The one or more engineered L. monocytogenes strains will be administered by any suitable route, such as intratumorally or intravenously. The one or more engineered L. monocytogenes strains can be administered in combination with the standard of care treatment for the cancer the subjects are diagnosed with. Treatment efficacy will then be established by measuring production of IL-2, IL-12 and/or CD40L in tumors, tumor volume, tumor size, disease progression, tumor metastases, and survival.

It is anticipated that the results will demonstrate that L. monocytogenes strains of the present technology produce IL-2, IL-12, and/or CD40L in a human tumor and selectively persist in a human tumor. It is further expected that the results will show that the L. monocytogenes strains of the present technology ameliorate, mitigate, or improve one or more symptoms of cancer including reducing tumor size, slowing tumor growth, reducing or preventing metastases, and increasing survival. Accordingly, these results will show that L. monocytogenes strains of the present technology are useful in methods and compositions for treating cancer in humans.

### REFERENCES

1 Haslam, A. & Prasad, V. Estimation of the Percentage of US Patients With Cancer Who Are Eligible for and Respond to Checkpoint Inhibitor Immunotherapy Drugs. JAMA Netw Open 2, e192535-e192535 (2019).
2. Carretero-González, A. et al. Analysis of response rate with anti-PD1/PDL1 antibodies in advanced solid tumors: A meta-analysis of randomized clinical trials (RCT). Journal of Clinical Oncology 35, 8706-8715 (2018).
3. Barnes, T. A. & Amir, E. HYPE or HOPE: the prognostic value of infiltrating immune cells in cancer. Br J Cancer 117, 451 (2017).
4. Li, F. et al. The association between CD8+ tumor-infiltrating lymphocytes and the clinical outcome of cancer immunotherapy: A systematic review and meta-analysis. eClinicalMedicine 41, 160 (2021).
5. Jiang, T., Zhou, C. & Ren, S. Role of IL-2 in cancer immunotherapy. Oncoimmunology 5, (2016).
6. Klapper, J. A. et al. High-dose Interleukin-2 for the Treatment of Metastatic Renal Cell Carcinoma: A Retrospective Analysis of Response and Survival in Patients Treated in the Surgery Branch at the National Cancer Institute Between 1986 and 2006. Cancer 113, 293 (2008).
7. Atkins, M. B. et al. High-dose recombinant interleukin 2 therapy for patients with metastatic melanoma: analysis of 270 patients treated between 1985 and 1993. J Clin Oncol 17, 2105-2116 (1999).
8. Pieper, A. A. et al. Combination of radiation therapy, bempegaldesleukin, and checkpoint blockade eradicates advanced solid tumors and metastases in mice. J Immunother Cancer 9, 2715 (2021).
9. Morrow, Z. T., Powers, Z. M. & Sauer, J. D. Listeria monocytogenes Cancer Vaccines: Bridging Innate and Adaptive Immunity. Current Clinical Microbiology Reports vol. 6 213-224 Preprint at https://doi.org/10.1007/s40588-019-00133-4 (2019).
10. Brocksted, D. G. et al. Listeria-based cancer vaccines that segregate immunogenicity from toxicity. Proc Natl Acad Sci USA 101, 13832-13837 (2004).
11. Le, D. T., Dubenksy, T. W. & Brockstedt, D. G. Clinical development of Listeria monocytogenes-based immunotherapies. Semin Oncol 39, 311-322 (2012).
12. Rolhion, N. & Cossart, P. How the study of Listeria monocytogenes has led to new concepts in biology. Future Microbiol 12, 621-638 (2017).
13. Lauer, P., Chow, M. Y. N., Loessner, M. J., Portnoy, D. A. & Calendar, R. Construction, characterization, and use of two Listeria monocytogenes site-specific phage integration vectors. J Bacteriol 184, 4177-4186 (2002).
14. Le, D. T. et al. A live-attenuated listeria vaccine (ANZ-100) and a live-attenuated listeria vaccine expressing mesothelin (CRS-207) for advanced cancers: Phase I studies of safety and immune induction. Clinical Cancer Research 18, 858-868 (2012).
15. Mengaud, J., Ohayon, H., Gounon, P., Mege, R. M. & Cossart, P. E-cadherin is the receptor for internalin, a surface protein required for entry of L. monocytogenes into epithelial cells. Cell 84, 923-932 (1996).
16. Shen, Y., Naujokas, M., Park, M. & Ireton, K. InIB-dependent internalization of Listeria is mediated by the Met receptor tyrosine kinase. Cell 103, 501-510 (2000).
17. Hamon, M. A., Ribet, D., Stavru, F. & Cossart, P. Listeriolysin O: The Swiss army knife of Listeria. Trends in Microbiology vol. 20 360-368 Preprint at https://doi.org/10.1016/j.tim.2012.04.006 (2012).
18. Glomski, I. J., Decatur, A. L. & Portnoy, D. A. Listeria monocytogenes Mutants That Fail to Compartmentalize Listerolysin O Activity Are Cytotoxic, Avirulent, and Unable to Evade Host Extracellular Defenses. Infect Immun 71, 6754-6765 (2003).
19. Morrow, Z. T., Powers, Z. M. & Sauer, J. D. Listeria monocytogenes Cancer Vaccines: Bridging Innate and Adaptive Immunity. Current Clinical Microbiology Reports vol. 6 213-224 Preprint at https://doi.org/10.1007/s40588-019-00133-4 (2019).
20. Kocks, C. et al. L. monocytogenes-induced actin assembly requires the actA gene product, a surface protein. Cell 68, 521-531 (1992).
21. Sauer, J. D. et al. Listeria monocytogenes engineered to activate the Nlrc4 inflammasome are severely attenuated and are poor inducers of protective immunity. Proc Natl Acad Sci USA 108, 12419-24 (2011).
22. Theisen, E. & Sauer, J. D. Listeria monocytogenes-induced cell death inhibits the generation of cell-mediated immunity. Infect Immun 85, e00733-16 (2017).
23. Theisen, E. et al. Cyclooxygenase-1 and -2 Play Contrasting Roles in Listeria-Stimulated Immunity. The Journal of Immunology 200, 3729-3738 (2018).
24. Deng, W. et al. Recombinant Listeria promotes tumor rejection by CD8+ T cell-dependent remodeling of the tumor microenvironment. Proc Natl Acad Sci USA 115, 8179-8184 (2018).
25. Crittenden, M. et al. Phase I study of safety and immunogenicity of ADU-623, a live-attenuated Listeria monocytogenes vaccine (Δ actA/ΔinlB) expressing EGFRVIII and N Y-ESO-1, in patients with WHO grade III/IV astrocytomas. Journal of Clinical Oncology 33, TPS3106-TPS3106 (2015).
26. Chávez-Arroyo, A. & Portnoy, D. A. Why is Listeria monocytogenes such a potent inducer of CD8+ T-cells? Cell Microbiol 22, e13175 (2020).
27. Chandra, D., Jahangir, A., Quispe-Tintaya, W., Einstein, M. H. & Gravekamp, C. Myeloid-derived suppressor cells have a central role in attenuated Listeria monocytogenes-based immunotherapy against metastatic breast cancer in young and old mice. Br J Cancer 108, 2281-2290 (2013).
28. Selvanesan, B. C. et al. Listeria delivers tetanus toxoid protein to pancreatic tumors and induces cancer cell death in mice. Sci Transl Med 14, 1600 (2022).
29. Rosenberg, S. A. IL-2: the first effective immunotherapy for human cancer. J Immunol 192, 5451-5458 (2014).
30. Canale, F. P. et al. Metabolic modulation of tumours with engineered bacteria for immunotherapy. 662 |Nature|598, (2021).
31. Sun, H. K., Castro, F., Paterson, Y. & Gravekamp, C. High efficacy of a Listeria-based vaccine against metastatic breast cancer reveals a dual mode of action. Cancer Res 69, 5860-5866 (2009).
32. Kim, S. H. et al. Mage-b vaccine delivered by recombinant Listeria monocytogenes is highly effective against breast cancer metastases. Br J Cancer 99, 741-749 (2008).
33. Jahangir, A. et al. Immunotherapy with Listeria reduces metastatic breast cancer in young and old mice through different mechanisms. Oncoimmunology 6, (2017).
34. Quispe-Tintaya, W. et al. Nontoxic radioactive Listeriaat is a highly effective therapy against metastatic pancreatic cancer. Proc Natl Acad Sci USA 110, 8668-8673 (2013).
35. Chandra, D. et al. 32-Phosphorus selectively delivered by listeria to pancreatic cancer demonstrates a strong therapeutic effect. Oncotarget 8, 20729-20740 (2017).
36. Theisen, E. & Sauer, J.-D. D. Listeria monocytogenes-Induced Cell Death Inhibits the Generation of Cell-Mediated Immunity. Infect Immun 85, (2017).
37. Monk, I. R., Gahan, C. G. M. & Hill, C. Tools for functional postgenomic analysis of Listeria monocytogenes. Appl Environ Microbiol 74, (2008).
38. Lebreton, A. & Cossart, P. RNA- and protein-mediated control of Listeria monocytogenes virulence gene expression. RNA Biol 14, 460 (2017).
39. Boyman, O. & Sprent, J. The role of interleukin-2 during homeostasis and activation of the immune system. Nature Reviews Immunology 2012 12:3 12, 180-190 (2012).
40. Morris, Z. S. et al. Tumor-specific inhibition of in situ vaccination by distant untreated tumor sites. Cancer Immunol Res 6, 825 (2018).
41. Vazquez-Lombardi, R. et al. Potent antitumour activity of interleukin-2-Fc fusion proteins requires Fc-mediated depletion of regulatory T-cells. Nat Commun 8, (2017).
42. Carmenate, T. et al. Human IL-2 mutein with higher antitumor efficacy than wild type IL-2. J Immunol 190, 6230-6238 (2013).
43. Habiba, U. e. et al. The multifaceted role of IL-12 in cancer. Advances in Cancer Biology—Metastasis 5, 100053 (2022).
44. Cirella, A. et al. Intratumoral Gene Transfer of mRNAs Encoding IL12 in Combination with Decoy-Resistant IL18 Improves Local and Systemic Antitumor Immunity. Cancer Immunol Res 11, 184-198 (2023).
45. Kohli, K., Pillarisetty, V. G. & Kim, T. S. Key chemokines direct migration of immune cells in solid tumors. Cancer Gene Therapy 2021 29:1 29, 10-21 (2021).
46. Lauer et al Construction, Characterization, and Use of Two Listeria monocytogenes Site-Specific Phage Integration Vectors. J. Bacteriology 2002.
47. Rakhmilevich A L, et al. Gene gun-mediated skin transfection with interleukin 12 gene results in regression of established primary and metastatic murine tumors. Proc Natl Acad Sci USA 1996 93:6291-6296.
48. Rakhmilevich A L, et al. Cytokine gene therapy of cancer using gene gun technology: superior antitumor activity of interleukin-12. Hum Gene Ther 1997 8:1303-1311.
49. Cheng E M, et al. Interleukin-12 as an in situ cancer vaccine component: a review. Cancer Immunol Immunother. 2022 September; 71 (9): 2057-2065.
50. Wigginton J M, et al. IL-12/IL-2 combination cytokine therapy for solid tumours: translation from bench to bedside. Expert Opin Biol Ther. 2002 June; 2 (5): 513-24.
51. Cheng E M, et al. Interleukin-12 as an in situ cancer vaccine component: a review. Cancer Immunol Immunother. 2022 September; 71 (9): 2057-2065.
52. Bullock, T. N. J., CD40 stimulation as a molecular adjuvant for cancer vaccines and other immunotherapies. Nature 2021.
53. Voeller J. Erbe A K, Slowinski J, Rasmussen K, Carlson P M, Hoefges A, VandenHeuvel S Stuckwisch A, Wang X, Gillies S D, Patel R, Farrell A, Maris J M, Hank J A, Morris Z S, Rakhmilevich A L, Sondel P M. Combined Innate and Adaptive Immunotherapeutic Approach Overcomes Resistance of Immunologically Cold Syngeneic Murine Neuroblastoma to Checkpoint Inhibition. J Immunother Cancer. 2019 Dec. 6; 7 (1): 344.
54. Aiken T J, et al. Mechanism of effective combination radio-immunotherapy against 9464D-GD2, an immunologically cold murine neuroblastoma. J Immunother Cancer. 2022 May; 10 (5): e004834.
55. Richards D M, Sefrin J P, Gieffers C, Hill O, Merz C. Concepts for agonistic targeting of CD40 in immuno-oncology. Hum Vaccin Immunother. 2020; 16 (2): 377-387.
56. Buhtoiarov I N, Sondel P M, Wigginton J M, Buhtoiarova T N, Yanke E M, Mahvi D A, Rakhmilevich A L. Antitumor Synergy of Cytotoxic Chemotherapy and Anti-CD40 Plus CpG-ODN Immunotherapy Through Repolarization of Tumor Associated Macrophages. Immunology. 132:226-239, 2011. PMCID: PMC3050446
57. Qu X, Felder M A R, Perez Horta Z, Sondel P M, Rakhmilevich A L. Antitumor effects of anti-CD40/CpG immunotherapy combined with gemcitabine or 5-fluorouracil chemotherapy in the B16 melanoma model. International Immunopharmacology, 17:1141-1147, 2013. PMCID: PMC3873769

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the appended claims. The present technology is to be limited only by the terms of the appended claims, along with the full scope of equivalents to which such claims are entitled. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

Each and every publication and patent mentioned in the above specification is herein incorporated by reference in its entirety for all purposes. Various modifications and variations of the described methods and system of the present technology will be apparent to those skilled in the art without departing from the scope and spirit of the present technology. Although the present technology has been described in connection with specific embodiments, the present technology as claimed should not be unduly limited to such specific embodiments. Indeed, various modifications of the described modes for carrying out the present technology which are obvious to those skilled in the art and in fields related thereto are intended to be within the scope of the following claims.

Sequence Listing

ActAN30-IL2 (SEQ ID NO: 1)
aggagagtgaaacccatggGtgggattaaatagatttatgcgtgcgatgatggtagttttcattactgccaactgcattacgattaaccccgac
ataatatttgcagcgCCAACATCTTCTTCCACTAAGAAGACTCAGCTACAACTTGAACACCTT
CTGTTGGACCTACAAATGATTCTAAATGGAATAAACAATTATAAAAATCCTAAATTA
ACTAGAATGCTCACGTTCAAATTCTACATGCCGAAAAAAGCAACCGAGTTAAAACA
TTTACAATGTCTTGAAGAAGAACTTAAACCATTGGAAGAAGTGCTTAACTTGGCCCA
AAGTAAAAATTTTCATTTACGTCCACGAGATTTAATTAGCAATATCAATGTCATTGT
ATTAGAATTAAAAGGTAGTGAAACCACATTTATGTGCGAATATGCTGATGAGACAG
CGACAATTGTTGAATTTTTAAACCGCTGGATTACATTTTGTCAATCAATTATCTCGAC
GTTAACGTGAGTCGACCTCGAGGGG
ActAN100-IL2 (SEQ ID NO: 2)
gaaggagagtgaaacccatggGtgggattaaatagatttatgcgtgcgatgatggtagttttcattactgccaactgcattacgattaacccc
gacataatatttgcagcgacagatagcgaagattccagtctaaacacagatgaatgggaagaagaaaaaacagaagagcagccaagcga
ggtaaatacgggaccaagatacgaaactgcacgtgaagtaagttcacgtgatattgaggaactagaaaaatcgaataaagtgaaaaatacg
aacaaagcagacctaatagcaatgttgaaagcaaaagcagagaaaggtggatccCCAACATCTTCTTCCACTAAGA
AGACTCAGCTACAACTTGAACACCTTCTGTTGGACCTACAAATGATTCTAAATGGAA
TAAACAATTATAAAAATCCTAAATTAACTAGAATGCTCACGTTCAAATTCTACATGC
CGAAAAAAGCAACCGAGTTAAAACATTTACAATGTCTTGAAGAAGAACTTAAACCA
TTGGAAGAAGTGCTTAACTTGGCCCAAAGTAAAAATTTTCATTTACGTCCACGAGAT
TTAATTAGCAATATCAATGTCATTGTATTAGAATTAAAAGGTAGTGAAACCACATTT
ATGTGCGAATATGCTGATGAGACAGCGACAATTGTTGAATTTTTAAACCGCTGGATT
ACATTTTGTCAATCAATTATCTCGACGTTAACGTGAGTCGACCTCGAGGGGGGG
phelp-hly5′ utr_fwd (SEQ ID NO: 3)
agggaacaaaagctgggtacCCATTATGCTTTGGCAGTTTATTC
phelp-hly5′ utr_rev (SEQ ID NO: 4)
agcctgacatGGGTTTCACTCTCCTTCTAC
hly-F (SEQ ID NO: 5)
TCAACCAGATGTTCTCCCTGTA
hly-R (SEQ ID NO: 6)
CACTGTAAGCCATTTCGTCATC
pIMK2 (SEQ ID NO: 7)
TTGGCAGCATCACCCGACGCACTTTGCGCCGAATAAATACCTGTGACGGAAGATCA
CTTCGCAGAATAAATAAATCCTGGTGTCCCTGTTGATACCGGGAAGCCCTGGGACGT
CGGGCCCTTTCGTCTTCAAGAATTAATTCCCAATTCCAGGCATCAAATAAAACGAAA
GGCTCAGTCGAAAGACTGGGCCTTTCGTTTTATCTGTTGTTTGTCGGTGAACGCTCTC
CTGAGTAGGACAAATCCGCCGGGAGCGGATTTGAACGTTGCGAAGCAACGGCCCGG
AGGGTGGCGGGCAGGACGCCCGCCATAAACTGCCAGGAATTAATTCCAGGCATCAA
ATAAAACGAAAGGCTCAGTCGAAAGACTGGGCCTTTCGTTTTATCTGTTGTTTGTCG
GTGAACGCTCTCCTGAGTAGGACAAATCCGCCGGGAGCGGATTTGAACGTTGCGAA
GCAACGGCCCGGAGGGTGGCGGGCAGGACGCCCGCCATAAACTGCCAGGAATTAAT
TCCAGGCATCAAATAAAACGAAAGGCTCAGTCGAAAGACTGGGCCTTTCGTTTTATC
TGTTGTTTGTCGGTGAACGCTCTCCTGAGTAGGACAAATCCGCCGGGAGCGGATTTG
AACGTTGCGAAGCAACGGCCCGGAGGGTGGCGGGCAGGACGCCCGCCATAAACTGC
CAGGAATTAATTCCAGGCATCAAATAAAACGAAAGGCTCAGTCGAAAGACTGGGCC
TTTCGTTTTATCTGTTGTTTGTCGGTGAACGCTCTCCTGAGTAGGACAAATCCGCCGG
GAGCGGATTTGAACGTTGCGAAGCAACGGCCCGGAGGGTGGCGGGCAGGACGCCC
GCCATAAACTGCCAGGAATTGGGGATCGGAATTCGAGCTCcattatgctttggcagtttattcttgacat
gtagtgagggggctggtataatcacatacggccgataaagcaagcatataatattgcgtttcatctttagaagcgaatttcgccaatattat
aattatcaaaagagaggggtggcaaacggtatttggcattattaggttaaaaaatgtagaaggagagtgaaacccatggaaaaGGATCCC
CCGGGCTGCAGGAATTCGATATCAAGCTTATCGATACCGTCGACCTCGAGGGGGGG
CCCGGTACCCAGCTTTTGTTCCCTTTAGTGAGGGTTAATTCCCAGCGAACCATTTGAG
GTGATAGGTAAGATTATACCGAGGTATGAAAACGAGAATTGGACCTTTACAGAATT
ACTCTATGAAGCGCCATATTTAAAAAGCTACCAAGACGAAGAGGATGAAGAGGATG
AGGAGGCAGATTGCCTTGAATATATTGACAATACTGATAAGATAATATATCTTTTAT
ATAGAAGATATCGCCGTATGTAAGGATTTCAGGGGGCAAGGCATAGGCAGCGCGCT
TATCAATATATCTATAGAATGGGCAAAGCATAAAAACTTGCATGGACTAATGCTTGA
AACCCAGGACAATAACCTTATAGCTTGTAAATTCTATCATAATTGTGGTTTCAAAAT
CGGCTCCGTCGATACTATGTTATACGCCAACTTTGAAAACAACTTTGAAAAAGCTGT
TTTCTGGTATTTAAGGTTTTAGAATGCAAGGAACAGTGAATTGGAGTTCGTCTTGTT
ATAATTAGCTTCTTGGGGTATCTTTAAATACTGTAGAAAAGAGGAAGGAAATAATA
AATGGCTAAAATGAGAATATCACCGGAATTGAAAAAACTGATCGAAAAATACCGCT
GCGTAAAAGATACGGAAGGAATGTCTCCTGCTAAGGTATATAAGCTGGTGGGAGAA
AATGAAAACCTATATTTAAAAATGACGGACAGCCGGTATAAAGGGACCACCTATGA
TGTGGAACGGGAAAAGGACATGATGCTATGGCTGGAAGGAAAGCTGCCTGTTCCAA
AGGTCCTGCACTTTGAACGGCATGATGGCTGGAGCAATCTGCTCATGAGTGAGGCCG
ATGGCGTCCTTTGCTCGGAAGAGTATGAAGATGAACAAAGCCCTGAAAAGATTATC
GAGCTGTATGCGGAGTGCATCAGGCTCTTTCACTCCATCGACATATCGGATTGTCCC
TATACGAATAGCTTAGACAGCCGCTTAGCCGAATTGGATTACTTACTGAATAACGAT
CTGGCCGATGTGGATTGCGAAAACTGGGAAGAAGACACTCCATTTAAAGATCCGCG
CGAGCTGTATGATTTTTTAAAGACGGAAAAGCCCGAAGAGGAACTTGTCTTTTCCCA
CGGCGACCTGGGAGACAGCAACATCTTTGTGAAAGATGGCAAAGTAAGTGGCTTTA
TTGATCTTGGGAGAAGCGGCAGGGCGGACAAGTGGTATGACATTGCCTTCTGCGTCC
GGTCGATCAGGGAGGATATCGGGGAAGAACAGTATGTCGAGCTATTTTTTGACTTAC
TGGGGATCAAGCCTGATTGGGAGAAAATAAAATATTATATTTTACTGGATGAATTGT
TTTAGTACCTAGATTTAGATGTCTAAAAAGCTTTAACTACAAGCTTTTTAGACATCTA
ATCTTTTCTGAAGTACATCCGCAACTGTCCATACTCTGATGTTTCATATGATCATCAT
AATTCTGTCTCATTATATAACATCCTCCATACCTTCTATTATAGAATACCATAAACTC
ATCTGGCAATTCATTTCGAGTCACGAAGAACGGAAAAACTGCCGGTTTTTATATTAC
AAATGTATTAAGTTTTTCTATTAACAAAAAACAATAGGTTTCCCATAGCGAAAGTTG
TTGATTAACGTTCACATCCCACTTACACTATAAAGGTTTACCCAGCAATACATCTCA
AGCCCTAAGAATACACGTTCGCTTTTCAACTGTTACAGAATTATTACAAATAGTTGG
TATAGTCCTCTTTAGCCTTTGGAGCTATTATCTCATCATTTGTTTTTTAGGTGAAAAC
TGGGTAAACTTAGTATTAATCAATATAAAATTAATTCTCAAATACTTAATTACGTAC
TGGGATTTTCTGAAAAAAAGATCTCCAAAAATAAACAGGTGGTGGTATTAATGAAG
ATAAAAAAATTAGCAAACGGTAAATATTGTGTTCGCCTACGTATAAAAGTCGATGGT
GAATGGAAAGAAAAGCGTTTGACAGATACAAGTGAAACAAACTTAATGTATAAAGC
ATCTAAATTATTAAAACAAGTTCAGCATGATAGTAGTTCTCTGAAAGAATGGAACTT
CAAAGAATTTTATACGCTATTCATGAAAACATTTAAAGATGGGAAAAGTAGTCAATC
TACTATTAATTTATACGATCTTGCTTATAATCAATTCGTTGATTATTTCGATGAAAAA
ATTAAATTTAATTCGATTGATGCGGTTCAATATCAACAATTTATTAATCATTTATCTG
TAGACTATGCAATATCCACTGTAGACACCAGACACCGCAAAATTAGAGCGATTTTTA
ACAAGGCTGTTCATTTAGGTTACATGAAGAAAAACCCCACTATAGGGGCTCATATAA
GCGGACAGGACGTAGCGAAAAATAAAGCACAATTTATGGAAACAGACAAAGTTCAT
TTACTATTAGAAGAACTTGCAAAATTTCATTCTATATCACGAGCAGTTATCTTTCTAG
CTGTCCAGACAGGCATGAGGTTCGAAGAAATTATTGCACTAACAAAGAAGGATATT
AATTTCACTAAACGTTCAATAACTGTGAATAAAGCTTGGGATTACAAGTACACTAAT
ACATTCATTGATACCAAAACAAAAAAATCACGAGTGATCTATATTGATAACTCTACC
GCTCAATATTTACATTCGTATTTAAATTGGCATACTGAATATATGAAGGAACATGCT
ATTAAGAATCCATTGATGTTATTATTCATCACTTACCACAATAAGCCAGTAGACAAC
GCGTCTTGTAATAAAGCTTTGAAGAAGATATGTAGTACAATCAATTCTGAACCAGTG
ACATTACACAAGCTACGACATACGCATACAGGCTTATGTGTAGAAGCGGGTATGGA
TATTATTTATGTAGCTGATAGGCTTGGTCATGATGACATTAATACAACATTAAAATA
CTATAGTCATCTAAGCTCTAATTTAAGACAACATAATCAGTCCAAAGTAGATGCTTT
TTTCACACTAAAAACAGATGAAAATACCACAAATTTTACCACAAATGCCACAAAAA
CAACGGAATAAAACGGGTATTATACGATATAAAAAAAACTCCAAAACATTCATCCG
CCCTTTAATATCAAGGCTTTTCAACGTTTTAGAGATTTCTTTACATTACTATTTAACG
TCCTGAGAGGGATTAACACACACTGATATAAAGCCATTTAGGATATATATACCACAA
ATAATACCACAAACATTTTATGTAATAATAAATATTATTTATTATTACATTGAAATA
AATATTCGTTATAAATAGTTTTTATATCAAGATGTTTTTTCTCAAGGTTTTTATAAAA
TGACTTTAATTCTTTTGTTTCAAGTAGTCCAGAGAAGATTTTTTCAACAGCGTTCTTC
TTTCCCTCCACGCATGCCTCTCGCCTGTCCCCTCAGTTCAGTAATTTCCTGCATTTGC
CTGTTTCCAGTCGGTAGATATTCCACAAAACAGCAGGGAAGCAGCGCTTTTCCGCTG
CATAACCCTGCTTCGGGGTCATTATAGCGATTTTTTCGGTATATCCATCCTTTTTCGC
ACGATATACAGGATTTTGCCAAAGGGTTCGTGTAGACTTTCCTTGGTGTATCCAACG
GCGTCAGCCGGGCAGGATAGGTGAAGTAGGCCCACCCGCGAGCGGGTGTTCCTTCT
TCACTGTCCCTTATTCGCACCTGGCGGTGCTCAACGGGAATCCTGCTCTGCGAGGCT
GGCCGGCTACCGCCGGCGTAACAGATGAGGGCAAGCGGCGGAGAATTACAACTTAT
ATCGTATGGGGCTGACTTCAGGTGCTACATTTGAAGAGATAAATTGCACTGAAATCT
AGAAATATTTTATCTGATTAATAAGATGATCTTCTTGAGATCGTTTTGGTCTGCGCGT
AATCTCTTGCTCTGAAAACGAAAAAACCGCCTTGCAGGGCGGTTTTTCGAAGGTTCT
CTGAGCTACCAACTCTTTGAACCGAGGTAACTGGCTTGGAGGAGCGCAGTCACCAA
AACTTGTCCTTTCAGTTTAGCCTTAACCGGCGCATGACTTCAAGACTAACTCCTCTAA
ATCAATTACCAGTGGCTGCTGCCAGTGGTGCTTTTGCATGTCTTTCCGGGTTGGACTC
AAGACGATAGTTACCGGATAAGGCGCAGCGGTCGGACTGAACGGGGGGTTCGTGCA
TACAGTCCAGCTTGGAGCGAACTGCCTACCCGGAACTGAGTGTCAGGCGTGGAATG
AGACAAACGCGGCCATAACAGCGGAATGACACCGGTAAACCGAAAGGCAGGAACA
GGAGAGCGCACGAGGGAGCCGCCAGGGGGAAACGCCTGGTATCTTTATAGTCCTGT
CGGGTTTCGCCACCACTGATTTGAGCGTCAGATTTCGTGATGCTTGTCAGGGGGGCG
GAGCCTATGGAAAAACGGCTTTGCCGCGGCCCTCTCACTTCCCTGTTAAGTATCTTC
CTGGCATCTTCCAGGAAATCTCCGCCCCGTTCGTAAGCCATTTCCGCTCGCCGCAGT
CGAACGACCGAGCGTAGCGAGTCAGTGAGCGAGGAAGCGGAATATATCCTGTATCA
CATATTCTGCTGACGCACCGGTGCAGCCTTTTTTCTCCTGCCACATGAAGCACTTCAC
TGACACCCTCATCAGTGCCAACATAGTAAGCCAGTATACACTCCGCTAGCGCTGATG
TCCGGCGGTGCTTTTGCCGTTACGCACCACCCCGTCAGTAGCTGAACAGGAGGGACA
GCTGATAGAAACAGAAGCCACTGGAGCACCTCAAAAACACCATCATACACTAAATC
AGTAAG
pIMK2-ActAN30-IL-2 (SEQ ID NO: 8)
TTGGCAGCATCACCCGACGCACTTTGCGCCGAATAAATACCTGTGACGGAAGATCA
CTTCGCAGAATAAATAAATCCTGGTGTCCCTGTTGATACCGGGAAGCCCTGGGACGT
CGGGCCCTTTCGTCTTCAAGAATTAATTCCCAATTCCAGGCATCAAATAAAACGAAA
GGCTCAGTCGAAAGACTGGGCCTTTCGTTTTATCTGTTGTTTGTCGGTGAACGCTCTC
CTGAGTAGGACAAATCCGCCGGGAGCGGATTTGAACGTTGCGAAGCAACGGCCCGG
AGGGTGGCGGGCAGGACGCCCGCCATAAACTGCCAGGAATTAATTCCAGGCATCAA
ATAAAACGAAAGGCTCAGTCGAAAGACTGGGCCTTTCGTTTTATCTGTTGTTTGTCG
GTGAACGCTCTCCTGAGTAGGACAAATCCGCCGGGAGCGGATTTGAACGTTGCGAA
GCAACGGCCCGGAGGGTGGCGGGCAGGACGCCCGCCATAAACTGCCAGGAATTAAT
TCCAGGCATCAAATAAAACGAAAGGCTCAGTCGAAAGACTGGGCCTTTCGTTTTATC
TGTTGTTTGTCGGTGAACGCTCTCCTGAGTAGGACAAATCCGCCGGGAGCGGATTTG
AACGTTGCGAAGCAACGGCCCGGAGGGTGGCGGGCAGGACGCCCGCCATAAACTGC
CAGGAATTAATTCCAGGCATCAAATAAAACGAAAGGCTCAGTCGAAAGACTGGGCC
TTTCGTTTTATCTGTTGTTTGTCGGTGAACGCTCTCCTGAGTAGGACAAATCCGCCGG
GAGCGGATTTGAACGTTGCGAAGCAACGGCCCGGAGGGTGGCGGGCAGGACGCCC
GCCATAAACTGCCAGGAATTGGGGATCGGAATTCGAGCTCcattatgctttggcagtttattcttgacat
gtagtgagggggctggtataatcacatacggccgataaagcaagcatataatattgcgtttcatctttagaagcgaatttcgccaatattat
aattatcaaaagagaggggtggcaaacggtatttggcattattaggttaaaaaatgtagaaggagagtgaaacccatggGtgggattaaata
gatttatgcgtgcgatgatggtagttttcattactgccaactgcattacgattaaccccgacataatatttgcagcgCCAACATCTTCTTC
CACTAAGAAGACTCAGCTACAACTTGAACACCTTCTGTTGGACCTACAAATGATTCT
AAATGGAATAAACAATTATAAAAATCCTAAATTAACTAGAATGCTCACGTTCAAATT
CTACATGCCGAAAAAAGCAACCGAGTTAAAACATTTACAATGTCTTGAAGAAGAAC
TTAAACCATTGGAAGAAGTGCTTAACTTGGCCCAAAGTAAAAATTTTCATTTACGTC
CACGAGATTTAATTAGCAATATCAATGTCATTGTATTAGAATTAAAAGGTAGTGAAA
CCACATTTATGTGCGAATATGCTGATGAGACAGCGACAATTGTTGAATTTTTAAACC
GCTGGATTACATTTTGTCAATCAATTATCTCGACGTTAACGTGAGTCGACCTCGAGG
GGGGGCCCGGTACCCAGCTTTTGTTCCCTTTAGTGAGGGTTAATTCCCAGCGAACCA
TTTGAGGTGATAGGTAAGATTATACCGAGGTATGAAAACGAGAATTGGACCTTTAC
AGAATTACTCTATGAAGCGCCATATTTAAAAAGCTACCAAGACGAAGAGGATGAAG
AGGATGAGGAGGCAGATTGCCTTGAATATATTGACAATACTGATAAGATAATATAT
CTTTTATATAGAAGATATCGCCGTATGTAAGGATTTCAGGGGGCAAGGCATAGGCA
GCGCGCTTATCAATATATCTATAGAATGGGCAAAGCATAAAAACTTGCATGGACTA
ATGCTTGAAACCCAGGACAATAACCTTATAGCTTGTAAATTCTATCATAATTGTGGT
TTCAAAATCGGCTCCGTCGATACTATGTTATACGCCAACTTTGAAAACAACTTTGAA
AAAGCTGTTTTCTGGTATTTAAGGTTTTAGAATGCAAGGAACAGTGAATTGGAGTTC
GTCTTGTTATAATTAGCTTCTTGGGGTATCTTTAAATACTGTAGAAAAGAGGAAGGA
AATAATAAATGGCTAAAATGAGAATATCACCGGAATTGAAAAAACTGATCGAAAAA
TACCGCTGCGTAAAAGATACGGAAGGAATGTCTCCTGCTAAGGTATATAAGCTGGT
GGGAGAAAATGAAAACCTATATTTAAAAATGACGGACAGCCGGTATAAAGGGACCA
CCTATGATGTGGAACGGGAAAAGGACATGATGCTATGGCTGGAAGGAAAGCTGCCT
GTTCCAAAGGTCCTGCACTTTGAACGGCATGATGGCTGGAGCAATCTGCTCATGAGT
GAGGCCGATGGCGTCCTTTGCTCGGAAGAGTATGAAGATGAACAAAGCCCTGAAAA
GATTATCGAGCTGTATGCGGAGTGCATCAGGCTCTTTCACTCCATCGACATATCGGA
TTGTCCCTATACGAATAGCTTAGACAGCCGCTTAGCCGAATTGGATTACTTACTGAA
TAACGATCTGGCCGATGTGGATTGCGAAAACTGGGAAGAAGACACTCCATTTAAAG
ATCCGCGCGAGCTGTATGATTTTTTAAAGACGGAAAAGCCCGAAGAGGAACTTGTCT
TTTCCCACGGCGACCTGGGAGACAGCAACATCTTTGTGAAAGATGGCAAAGTAAGT
GGCTTTATTGATCTTGGGAGAAGCGGCAGGGCGGACAAGTGGTATGACATTGCCTTC
TGCGTCCGGTCGATCAGGGAGGATATCGGGGAAGAACAGTATGTCGAGCTATTTTTT
GACTTACTGGGGATCAAGCCTGATTGGGAGAAAATAAAATATTATATTTTACTGGAT
GAATTGTTTTAGTACCTAGATTTAGATGTCTAAAAAGCTTTAACTACAAGCTTTTTAG
ACATCTAATCTTTTCTGAAGTACATCCGCAACTGTCCATACTCTGATGTTTCATATGA
TCATCATAATTCTGTCTCATTATATAACATCCTCCATACCTTCTATTATAGAATACCA
TAAACTCATCTGGCAATTCATTTCGAGTCACGAAGAACGGAAAAACTGCCGGTTTTT
ATATTACAAATGTATTAAGTTTTTCTATTAACAAAAAACAATAGGTTTCCCATAGCG
AAAGTTGTTGATTAACGTTCACATCCCACTTACACTATAAAGGTTTACCCAGCAATA
CATCTCAAGCCCTAAGAATACACGTTCGCTTTTCAACTGTTACAGAATTATTACAAA
TAGTTGGTATAGTCCTCTTTAGCCTTTGGAGCTATTATCTCATCATTTGTTTTTTAGGT
GAAAACTGGGTAAACTTAGTATTAATCAATATAAAATTAATTCTCAAATACTTAATT
ACGTACTGGGATTTTCTGAAAAAAAGATCTCCAAAAATAAACAGGTGGTGGTATTA
ATGAAGATAAAAAAATTAGCAAACGGTAAATATTGTGTTCGCCTACGTATAAAAGT
CGATGGTGAATGGAAAGAAAAGCGTTTGACAGATACAAGTGAAACAAACTTAATGT
ATAAAGCATCTAAATTATTAAAACAAGTTCAGCATGATAGTAGTTCTCTGAAAGAAT
GGAACTTCAAAGAATTTTATACGCTATTCATGAAAACATTTAAAGATGGGAAAAGT
AGTCAATCTACTATTAATTTATACGATCTTGCTTATAATCAATTCGTTGATTATTTCG
ATGAAAAAATTAAATTTAATTCGATTGATGCGGTTCAATATCAACAATTTATTAATC
ATTTATCTGTAGACTATGCAATATCCACTGTAGACACCAGACACCGCAAAATTAGAG
CGATTTTTAACAAGGCTGTTCATTTAGGTTACATGAAGAAAAACCCCACTATAGGGG
CTCATATAAGCGGACAGGACGTAGCGAAAAATAAAGCACAATTTATGGAAACAGAC
AAAGTTCATTTACTATTAGAAGAACTTGCAAAATTTCATTCTATATCACGAGCAGTT
ATCTTTCTAGCTGTCCAGACAGGCATGAGGTTCGAAGAAATTATTGCACTAACAAAG
AAGGATATTAATTTCACTAAACGTTCAATAACTGTGAATAAAGCTTGGGATTACAAG
TACACTAATACATTCATTGATACCAAAACAAAAAAATCACGAGTGATCTATATTGAT
AACTCTACCGCTCAATATTTACATTCGTATTTAAATTGGCATACTGAATATATGAAG
GAACATGCTATTAAGAATCCATTGATGTTATTATTCATCACTTACCACAATAAGCCA
GTAGACAACGCGTCTTGTAATAAAGCTTTGAAGAAGATATGTAGTACAATCAATTCT
GAACCAGTGACATTACACAAGCTACGACATACGCATACAGGCTTATGTGTAGAAGC
GGGTATGGATATTATTTATGTAGCTGATAGGCTTGGTCATGATGACATTAATACAAC
ATTAAAATACTATAGTCATCTAAGCTCTAATTTAAGACAACATAATCAGTCCAAAGT
AGATGCTTTTTTCACACTAAAAACAGATGAAAATACCACAAATTTTACCACAAATGC
CACAAAAACAACGGAATAAAACGGGTATTATACGATATAAAAAAAACTCCAAAACA
TTCATCCGCCCTTTAATATCAAGGCTTTTCAACGTTTTAGAGATTTCTTTACATTACT
ATTTAACGTCCTGAGAGGGATTAACACACACTGATATAAAGCCATTTAGGATATATA
TACCACAAATAATACCACAAACATTTTATGTAATAATAAATATTATTTATTATTACAT
TGAAATAAATATTCGTTATAAATAGTTTTTATATCAAGATGTTTTTTCTCAAGGTTTT
TATAAAATGACTTTAATTCTTTTGTTTCAAGTAGTCCAGAGAAGATTTTTTCAACAGC
GTTCTTCTTTCCCTCCACGCATGCCTCTCGCCTGTCCCCTCAGTTCAGTAATTTCCTGC
ATTTGCCTGTTTCCAGTCGGTAGATATTCCACAAAACAGCAGGGAAGCAGCGCTTTT
CCGCTGCATAACCCTGCTTCGGGGTCATTATAGCGATTTTTTCGGTATATCCATCCTT
TTTCGCACGATATACAGGATTTTGCCAAAGGGTTCGTGTAGACTTTCCTTGGTGTATC
CAACGGCGTCAGCCGGGCAGGATAGGTGAAGTAGGCCCACCCGCGAGCGGGTGTTC
CTTCTTCACTGTCCCTTATTCGCACCTGGCGGTGCTCAACGGGAATCCTGCTCTGCGA
GGCTGGCCGGCTACCGCCGGCGTAACAGATGAGGGCAAGCGGCGGAGAATTACAAC
TTATATCGTATGGGGCTGACTTCAGGTGCTACATTTGAAGAGATAAATTGCACTGAA
ATCTAGAAATATTTTATCTGATTAATAAGATGATCTTCTTGAGATCGTTTTGGTCTGC
GCGTAATCTCTTGCTCTGAAAACGAAAAAACCGCCTTGCAGGGCGGTTTTTCGAAGG
TTCTCTGAGCTACCAACTCTTTGAACCGAGGTAACTGGCTTGGAGGAGCGCAGTCAC
CAAAACTTGTCCTTTCAGTTTAGCCTTAACCGGCGCATGACTTCAAGACTAACTCCTC
TAAATCAATTACCAGTGGCTGCTGCCAGTGGTGCTTTTGCATGTCTTTCCGGGTTGGA
CTCAAGACGATAGTTACCGGATAAGGCGCAGCGGTCGGACTGAACGGGGGGTTCGT
GCATACAGTCCAGCTTGGAGCGAACTGCCTACCCGGAACTGAGTGTCAGGCGTGGA
ATGAGACAAACGCGGCCATAACAGCGGAATGACACCGGTAAACCGAAAGGCAGGA
ACAGGAGAGCGCACGAGGGAGCCGCCAGGGGGAAACGCCTGGTATCTTTATAGTCC
TGTCGGGTTTCGCCACCACTGATTTGAGCGTCAGATTTCGTGATGCTTGTCAGGGGG
GCGGAGCCTATGGAAAAACGGCTTTGCCGCGGCCCTCTCACTTCCCTGTTAAGTATC
TTCCTGGCATCTTCCAGGAAATCTCCGCCCCGTTCGTAAGCCATTTCCGCTCGCCGCA
GTCGAACGACCGAGCGTAGCGAGTCAGTGAGCGAGGAAGCGGAATATATCCTGTAT
CACATATTCTGCTGACGCACCGGTGCAGCCTTTTTTCTCCTGCCACATGAAGCACTTC
ACTGACACCCTCATCAGTGCCAACATAGTAAGCCAGTATACACTCCGCTAGCGCTGA
TGTCCGGCGGTGCTTTTGCCGTTACGCACCACCCCGTCAGTAGCTGAACAGGAGGGA
CAGCTGATAGAAACAGAAGCCACTGGAGCACCTCAAAAACACCATCATACACTAAA
TCAGTAAG
pIMK2-ActAN100-IL-2 (SEQ ID NO: 9)
TTGGCAGCATCACCCGACGCACTTTGCGCCGAATAAATACCTGTGACGGAAGATCA
CTTCGCAGAATAAATAAATCCTGGTGTCCCTGTTGATACCGGGAAGCCCTGGGACGT
CGGGCCCTTTCGTCTTCAAGAATTAATTCCCAATTCCAGGCATCAAATAAAACGAAA
GGCTCAGTCGAAAGACTGGGCCTTTCGTTTTATCTGTTGTTTGTCGGTGAACGCTCTC
CTGAGTAGGACAAATCCGCCGGGAGCGGATTTGAACGTTGCGAAGCAACGGCCCGG
AGGGTGGCGGGCAGGACGCCCGCCATAAACTGCCAGGAATTAATTCCAGGCATCAA
ATAAAACGAAAGGCTCAGTCGAAAGACTGGGCCTTTCGTTTTATCTGTTGTTTGTCG
GTGAACGCTCTCCTGAGTAGGACAAATCCGCCGGGAGCGGATTTGAACGTTGCGAA
GCAACGGCCCGGAGGGTGGCGGGCAGGACGCCCGCCATAAACTGCCAGGAATTAAT
TCCAGGCATCAAATAAAACGAAAGGCTCAGTCGAAAGACTGGGCCTTTCGTTTTATC
TGTTGTTTGTCGGTGAACGCTCTCCTGAGTAGGACAAATCCGCCGGGAGCGGATTTG
AACGTTGCGAAGCAACGGCCCGGAGGGTGGCGGGCAGGACGCCCGCCATAAACTGC
CAGGAATTAATTCCAGGCATCAAATAAAACGAAAGGCTCAGTCGAAAGACTGGGCC
TTTCGTTTTATCTGTTGTTTGTCGGTGAACGCTCTCCTGAGTAGGACAAATCCGCCGG
GAGCGGATTTGAACGTTGCGAAGCAACGGCCCGGAGGGTGGCGGGCAGGACGCCC
GCCATAAACTGCCAGGAATTGGGGATCGGAATTCGAGCTCcattatgctttggcagtttattcttgacat
gtagtgagggggctggtataatcacatacggccgataaagcaagcatataatattgcgtttcatctttagaagcgaatttcgccaatattat
aattatcaaaagagaggggtggcaaacggtatttggcattattaggttaaaaaatgtagaaggagagtgaaacccatggGgggattaaatag
atttatgcgtgcgatgatggtagttttcattactgccaactgcattacgattaaccccgacataatatttgcagcgacagatagcgaagatt
ccagtctaaacacagatgaatgggaagaagaaaaaacagaagagcagccaagcgaggtaaatacgggaccaagatacgaaactgcacgtgaa
gtaagttcacgtgatattgaggaactagaaaaatcgaataaagtgaaaaatacgaacaaagcagacctaatagcaatgttgaaagcaaaagc
agagaaaggtggatccCCAACATCTTCTTCCACTAAGAAGACTCAGCTACAACTTGAACACCT
TCTGTTGGACCTACAAATGATTCTAAATGGAATAAACAATTATAAAAATCCTAAATT
AACTAGAATGCTCACGTTCAAATTCTACATGCCGAAAAAAGCAACCGAGTTAAAAC
ATTTACAATGTCTTGAAGAAGAACTTAAACCATTGGAAGAAGTGCTTAACTTGGCCC
AAAGTAAAAATTTTCATTTACGTCCACGAGATTTAATTAGCAATATCAATGTCATTG
TATTAGAATTAAAAGGTAGTGAAACCACATTTATGTGCGAATATGCTGATGAGACA
GCGACAATTGTTGAATTTTTAAACCGCTGGATTACATTTTGTCAATCAATTATCTCGA
CGTTAACGTGAGTCGACCTCGAGGGGGGGCCCGGTACCCAGCTTTTGTTCCCTTTAG
TGAGGGTTAATTCCCAGCGAACCATTTGAGGTGATAGGTAAGATTATACCGAGGTAT
GAAAACGAGAATTGGACCTTTACAGAATTACTCTATGAAGCGCCATATTTAAAAAG
CTACCAAGACGAAGAGGATGAAGAGGATGAGGAGGCAGATTGCCTTGAATATATTG
ACAATACTGATAAGATAATATATCTTTTATATAGAAGATATCGCCGTATGTAAGGAT
TTCAGGGGGCAAGGCATAGGCAGCGCGCTTATCAATATATCTATAGAATGGGCAAA
GCATAAAAACTTGCATGGACTAATGCTTGAAACCCAGGACAATAACCTTATAGCTTG
TAAATTCTATCATAATTGTGGTTTCAAAATCGGCTCCGTCGATACTATGTTATACGCC
AACTTTGAAAACAACTTTGAAAAAGCTGTTTTCTGGTATTTAAGGTTTTAGAATGCA
AGGAACAGTGAATTGGAGTTCGTCTTGTTATAATTAGCTTCTTGGGGTATCTTTAAAT
ACTGTAGAAAAGAGGAAGGAAATAATAAATGGCTAAAATGAGAATATCACCGGAA
TTGAAAAAACTGATCGAAAAATACCGCTGCGTAAAAGATACGGAAGGAATGTCTCC
TGCTAAGGTATATAAGCTGGTGGGAGAAAATGAAAACCTATATTTAAAAATGACGG
ACAGCCGGTATAAAGGGACCACCTATGATGTGGAACGGGAAAAGGACATGATGCTA
TGGCTGGAAGGAAAGCTGCCTGTTCCAAAGGTCCTGCACTTTGAACGGCATGATGGC
TGGAGCAATCTGCTCATGAGTGAGGCCGATGGCGTCCTTTGCTCGGAAGAGTATGAA
GATGAACAAAGCCCTGAAAAGATTATCGAGCTGTATGCGGAGTGCATCAGGCTCTTT
CACTCCATCGACATATCGGATTGTCCCTATACGAATAGCTTAGACAGCCGCTTAGCC
GAATTGGATTACTTACTGAATAACGATCTGGCCGATGTGGATTGCGAAAACTGGGA
AGAAGACACTCCATTTAAAGATCCGCGCGAGCTGTATGATTTTTTAAAGACGGAAA
AGCCCGAAGAGGAACTTGTCTTTTCCCACGGCGACCTGGGAGACAGCAACATCTTTG
TGAAAGATGGCAAAGTAAGTGGCTTTATTGATCTTGGGAGAAGCGGCAGGGCGGAC
AAGTGGTATGACATTGCCTTCTGCGTCCGGTCGATCAGGGAGGATATCGGGGAAGA
ACAGTATGTCGAGCTATTTTTTGACTTACTGGGGATCAAGCCTGATTGGGAGAAAAT
AAAATATTATATTTTACTGGATGAATTGTTTTAGTACCTAGATTTAGATGTCTAAAAA
GCTTTAACTACAAGCTTTTTAGACATCTAATCTTTTCTGAAGTACATCCGCAACTGTC
CATACTCTGATGTTTCATATGATCATCATAATTCTGTCTCATTATATAACATCCTCCA
TACCTTCTATTATAGAATACCATAAACTCATCTGGCAATTCATTTCGAGTCACGAAG
AACGGAAAAACTGCCGGTTTTTATATTACAAATGTATTAAGTTTTTCTATTAACAAA
AAACAATAGGTTTCCCATAGCGAAAGTTGTTGATTAACGTTCACATCCCACTTACAC
TATAAAGGTTTACCCAGCAATACATCTCAAGCCCTAAGAATACACGTTCGCTTTTCA
ACTGTTACAGAATTATTACAAATAGTTGGTATAGTCCTCTTTAGCCTTTGGAGCTATT
ATCTCATCATTTGTTTTTTAGGTGAAAACTGGGTAAACTTAGTATTAATCAATATAAA
ATTAATTCTCAAATACTTAATTACGTACTGGGATTTTCTGAAAAAAAGATCTCCAAA
AATAAACAGGTGGTGGTATTAATGAAGATAAAAAAATTAGCAAACGGTAAATATTG
TGTTCGCCTACGTATAAAAGTCGATGGTGAATGGAAAGAAAAGCGTTTGACAGATA
CAAGTGAAACAAACTTAATGTATAAAGCATCTAAATTATTAAAACAAGTTCAGCAT
GATAGTAGTTCTCTGAAAGAATGGAACTTCAAAGAATTTTATACGCTATTCATGAAA
ACATTTAAAGATGGGAAAAGTAGTCAATCTACTATTAATTTATACGATCTTGCTTAT
AATCAATTCGTTGATTATTTCGATGAAAAAATTAAATTTAATTCGATTGATGCGGTTC
AATATCAACAATTTATTAATCATTTATCTGTAGACTATGCAATATCCACTGTAGACA
CCAGACACCGCAAAATTAGAGCGATTTTTAACAAGGCTGTTCATTTAGGTTACATGA
AGAAAAACCCCACTATAGGGGCTCATATAAGCGGACAGGACGTAGCGAAAAATAA
AGCACAATTTATGGAAACAGACAAAGTTCATTTACTATTAGAAGAACTTGCAAAATT
TCATTCTATATCACGAGCAGTTATCTTTCTAGCTGTCCAGACAGGCATGAGGTTCGA
AGAAATTATTGCACTAACAAAGAAGGATATTAATTTCACTAAACGTTCAATAACTGT
GAATAAAGCTTGGGATTACAAGTACACTAATACATTCATTGATACCAAAACAAAAA
AATCACGAGTGATCTATATTGATAACTCTACCGCTCAATATTTACATTCGTATTTAAA
TTGGCATACTGAATATATGAAGGAACATGCTATTAAGAATCCATTGATGTTATTATT
CATCACTTACCACAATAAGCCAGTAGACAACGCGTCTTGTAATAAAGCTTTGAAGAA
GATATGTAGTACAATCAATTCTGAACCAGTGACATTACACAAGCTACGACATACGCA
TACAGGCTTATGTGTAGAAGCGGGTATGGATATTATTTATGTAGCTGATAGGCTTGG
TCATGATGACATTAATACAACATTAAAATACTATAGTCATCTAAGCTCTAATTTAAG
ACAACATAATCAGTCCAAAGTAGATGCTTTTTTCACACTAAAAACAGATGAAAATAC
CACAAATTTTACCACAAATGCCACAAAAACAACGGAATAAAACGGGTATTATACGA
TATAAAAAAAACTCCAAAACATTCATCCGCCCTTTAATATCAAGGCTTTTCAACGTT
TTAGAGATTTCTTTACATTACTATTTAACGTCCTGAGAGGGATTAACACACACTGAT
ATAAAGCCATTTAGGATATATATACCACAAATAATACCACAAACATTTTATGTAATA
ATAAATATTATTTATTATTACATTGAAATAAATATTCGTTATAAATAGTTTTTATATC
AAGATGTTTTTTCTCAAGGTTTTTATAAAATGACTTTAATTCTTTTGTTTCAAGTAGT
CCAGAGAAGATTTTTTCAACAGCGTTCTTCTTTCCCTCCACGCATGCCTCTCGCCTGT
CCCCTCAGTTCAGTAATTTCCTGCATTTGCCTGTTTCCAGTCGGTAGATATTCCACAA
AACAGCAGGGAAGCAGCGCTTTTCCGCTGCATAACCCTGCTTCGGGGTCATTATAGC
GATTTTTTCGGTATATCCATCCTTTTTCGCACGATATACAGGATTTTGCCAAAGGGTT
CGTGTAGACTTTCCTTGGTGTATCCAACGGCGTCAGCCGGGCAGGATAGGTGAAGTA
GGCCCACCCGCGAGCGGGTGTTCCTTCTTCACTGTCCCTTATTCGCACCTGGCGGTG
CTCAACGGGAATCCTGCTCTGCGAGGCTGGCCGGCTACCGCCGGCGTAACAGATGA
GGGCAAGCGGCGGAGAATTACAACTTATATCGTATGGGGCTGACTTCAGGTGCTAC
ATTTGAAGAGATAAATTGCACTGAAATCTAGAAATATTTTATCTGATTAATAAGATG
ATCTTCTTGAGATCGTTTTGGTCTGCGCGTAATCTCTTGCTCTGAAAACGAAAAAAC
CGCCTTGCAGGGCGGTTTTTCGAAGGTTCTCTGAGCTACCAACTCTTTGAACCGAGG
TAACTGGCTTGGAGGAGCGCAGTCACCAAAACTTGTCCTTTCAGTTTAGCCTTAACC
GGCGCATGACTTCAAGACTAACTCCTCTAAATCAATTACCAGTGGCTGCTGCCAGTG
GTGCTTTTGCATGTCTTTCCGGGTTGGACTCAAGACGATAGTTACCGGATAAGGCGC
AGCGGTCGGACTGAACGGGGGGTTCGTGCATACAGTCCAGCTTGGAGCGAACTGCC
TACCCGGAACTGAGTGTCAGGCGTGGAATGAGACAAACGCGGCCATAACAGCGGAA
TGACACCGGTAAACCGAAAGGCAGGAACAGGAGAGCGCACGAGGGAGCCGCCAGG
GGGAAACGCCTGGTATCTTTATAGTCCTGTCGGGTTTCGCCACCACTGATTTGAGCG
TCAGATTTCGTGATGCTTGTCAGGGGGGCGGAGCCTATGGAAAAACGGCTTTGCCGC
GGCCCTCTCACTTCCCTGTTAAGTATCTTCCTGGCATCTTCCAGGAAATCTCCGCCCC
GTTCGTAAGCCATTTCCGCTCGCCGCAGTCGAACGACCGAGCGTAGCGAGTCAGTG
AGCGAGGAAGCGGAATATATCCTGTATCACATATTCTGCTGACGCACCGGTGCAGCC
TTTTTTCTCCTGCCACATGAAGCACTTCACTGACACCCTCATCAGTGCCAACATAGTA
AGCCAGTATACACTCCGCTAGCGCTGATGTCCGGCGGTGCTTTTGCCGTTACGCACC
ACCCCGTCAGTAGCTGAACAGGAGGGACAGCTGATAGAAACAGAAGCCACTGGAG
CACCTCAAAAACACCATCATACACTAAATCAGTAAG
pIMK2-hly5′UTR-ActAN100-IL2 (SEQ ID NO: 10)
TTGGCAGCATCACCCGACGCACTTTGCGCCGAATAAATACCTGTGACGGAAGATCA
CTTCGCAGAATAAATAAATCCTGGTGTCCCTGTTGATACCGGGAAGCCCTGGGACGT
CGGGCCCTTTCGTCTTCAAGAATTAATTCCCAATTCCAGGCATCAAATAAAACGAAA
GGCTCAGTCGAAAGACTGGGCCTTTCGTTTTATCTGTTGTTTGTCGGTGAACGCTCTC
CTGAGTAGGACAAATCCGCCGGGAGCGGATTTGAACGTTGCGAAGCAACGGCCCGG
AGGGTGGCGGGCAGGACGCCCGCCATAAACTGCCAGGAATTAATTCCAGGCATCAA
ATAAAACGAAAGGCTCAGTCGAAAGACTGGGCCTTTCGTTTTATCTGTTGTTTGTCG
GTGAACGCTCTCCTGAGTAGGACAAATCCGCCGGGAGCGGATTTGAACGTTGCGAA
GCAACGGCCCGGAGGGTGGCGGGCAGGACGCCCGCCATAAACTGCCAGGAATTAAT
TCCAGGCATCAAATAAAACGAAAGGCTCAGTCGAAAGACTGGGCCTTTCGTTTTATC
TGTTGTTTGTCGGTGAACGCTCTCCTGAGTAGGACAAATCCGCCGGGAGCGGATTTG
AACGTTGCGAAGCAACGGCCCGGAGGGTGGCGGGCAGGACGCCCGCCATAAACTGC
CAGGAATTAATTCCAGGCATCAAATAAAACGAAAGGCTCAGTCGAAAGACTGGGCC
TTTCGTTTTATCTGTTGTTTGTCGGTGAACGCTCTCCTGAGTAGGACAAATCCGCCGG
GAGCGGATTTGAACGTTGCGAAGCAACGGCCCGGAGGGTGGCGGGCAGGACGCCC
GCCATAAACTGCCAGGAATTGGGGATCGGAATTCGAGCTCcattatgctttggcagtttattcttgacat
gtagtgagggggctggtataatcacatacggccgataaagcaagcatataatattgcgtttcatctttagaagcgaatttcgccaatattat
aattatcaaaagagaggggtggcaaacggtatttggcattattaggttaaaaaatgtagaaggagagtgaaacccatgGATAAAGCAA
GCATATAATATTGCGTGTAGAAGGAGAGTGAAACCCGTGGGATTAAATAGATTTAT
GCGTGCGATGATGGTAGTTTTCATTACTGCCAACTGCATTACGATTAACCCCGACAT
AATATTTGCAGCGACAGATAGCGAAGATTCCAGTCTAAACACAGATGAATGGGAAG
AAGAAAAAACAGAAGAGCAGCCAAGCGAGGTAAATACGGGACCAAGATACGAAAC
TGCACGTGAAGTAAGTTCACGTGATATTGAGGAACTAGAAAAATCGAATAAAGTGA
AAAATACGAACAAAGCAGACCTAATAGCAATGTTGAAAGCAAAAGCAGAGAAAGG
TGGATCCCCAACATCTTCTTCCACTAAGAAGACTCAGCTACAACTTGAACACCTTCT
GTTGGACCTACAAATGATTCTAAATGGAATAAACAATTATAAAAATCCTAAATTAAC
TAGAATGCTCACGTTCAAATTCTACATGCCGAAAAAAGCAACCGAGTTAAAACATTT
ACAATGTCTTGAAGAAGAACTTAAACCATTGGAAGAAGTGCTTAACTTGGCCCAAA
GTAAAAATTTTCATTTACGTCCACGAGATTTAATTAGCAATATCAATGTCATTGTATT
AGAATTAAAAGGTAGTGAAACCACATTTATGTGCGAATATGCTGATGAGACAGCGA
CAATTGTTGAATTTTTAAACCGCTGGATTACATTTTGTCAATCAATTATCTCGACGTT
AACGTGAGTCGACCTCGAGGGGGGGCCCGGTACCCAGCTTTTGTTCCCTTTAGTGAG
GGTTAATTCCCAGCGAACCATTTGAGGTGATAGGTAAGATTATACCGAGGTATGAA
AACGAGAATTGGACCTTTACAGAATTACTCTATGAAGCGCCATATTTAAAAAGCTAC
CAAGACGAAGAGGATGAAGAGGATGAGGAGGCAGATTGCCTTGAATATATTGACAA
TACTGATAAGATAATATATCTTTTATATAGAAGATATCGCCGTATGTAAGGATTTCA
GGGGGCAAGGCATAGGCAGCGCGCTTATCAATATATCTATAGAATGGGCAAAGCAT
AAAAACTTGCATGGACTAATGCTTGAAACCCAGGACAATAACCTTATAGCTTGTAAA
TTCTATCATAATTGTGGTTTCAAAATCGGCTCCGTCGATACTATGTTATACGCCAACT
TTGAAAACAACTTTGAAAAAGCTGTTTTCTGGTATTTAAGGTTTTAGAATGCAAGGA
ACAGTGAATTGGAGTTCGTCTTGTTATAATTAGCTTCTTGGGGTATCTTTAAATACTG
TAGAAAAGAGGAAGGAAATAATAAATGGCTAAAATGAGAATATCACCGGAATTGA
AAAAACTGATCGAAAAATACCGCTGCGTAAAAGATACGGAAGGAATGTCTCCTGCT
AAGGTATATAAGCTGGTGGGAGAAAATGAAAACCTATATTTAAAAATGACGGACAG
CCGGTATAAAGGGACCACCTATGATGTGGAACGGGAAAAGGACATGATGCTATGGC
TGGAAGGAAAGCTGCCTGTTCCAAAGGTCCTGCACTTTGAACGGCATGATGGCTGG
AGCAATCTGCTCATGAGTGAGGCCGATGGCGTCCTTTGCTCGGAAGAGTATGAAGAT
GAACAAAGCCCTGAAAAGATTATCGAGCTGTATGCGGAGTGCATCAGGCTCTTTCAC
TCCATCGACATATCGGATTGTCCCTATACGAATAGCTTAGACAGCCGCTTAGCCGAA
TTGGATTACTTACTGAATAACGATCTGGCCGATGTGGATTGCGAAAACTGGGAAGA
AGACACTCCATTTAAAGATCCGCGCGAGCTGTATGATTTTTTAAAGACGGAAAAGCC
CGAAGAGGAACTTGTCTTTTCCCACGGCGACCTGGGAGACAGCAACATCTTTGTGAA
AGATGGCAAAGTAAGTGGCTTTATTGATCTTGGGAGAAGCGGCAGGGCGGACAAGT
GGTATGACATTGCCTTCTGCGTCCGGTCGATCAGGGAGGATATCGGGGAAGAACAG
TATGTCGAGCTATTTTTTGACTTACTGGGGATCAAGCCTGATTGGGAGAAAATAAAA
TATTATATTTTACTGGATGAATTGTTTTAGTACCTAGATTTAGATGTCTAAAAAGCTT
TAACTACAAGCTTTTTAGACATCTAATCTTTTCTGAAGTACATCCGCAACTGTCCATA
CTCTGATGTTTCATATGATCATCATAATTCTGTCTCATTATATAACATCCTCCATACC
TTCTATTATAGAATACCATAAACTCATCTGGCAATTCATTTCGAGTCACGAAGAACG
GAAAAACTGCCGGTTTTTATATTACAAATGTATTAAGTTTTTCTATTAACAAAAAAC
AATAGGTTTCCCATAGCGAAAGTTGTTGATTAACGTTCACATCCCACTTACACTATA
AAGGTTTACCCAGCAATACATCTCAAGCCCTAAGAATACACGTTCGCTTTTCAACTG
TTACAGAATTATTACAAATAGTTGGTATAGTCCTCTTTAGCCTTTGGAGCTATTATCT
CATCATTTGTTTTTTAGGTGAAAACTGGGTAAACTTAGTATTAATCAATATAAAATT
AATTCTCAAATACTTAATTACGTACTGGGATTTTCTGAAAAAAAGATCTCCAAAAAT
AAACAGGTGGTGGTATTAATGAAGATAAAAAAATTAGCAAACGGTAAATATTGTGT
TCGCCTACGTATAAAAGTCGATGGTGAATGGAAAGAAAAGCGTTTGACAGATACAA
GTGAAACAAACTTAATGTATAAAGCATCTAAATTATTAAAACAAGTTCAGCATGATA
GTAGTTCTCTGAAAGAATGGAACTTCAAAGAATTTTATACGCTATTCATGAAAACAT
TTAAAGATGGGAAAAGTAGTCAATCTACTATTAATTTATACGATCTTGCTTATAATC
AATTCGTTGATTATTTCGATGAAAAAATTAAATTTAATTCGATTGATGCGGTTCAAT
ATCAACAATTTATTAATCATTTATCTGTAGACTATGCAATATCCACTGTAGACACCA
GACACCGCAAAATTAGAGCGATTTTTAACAAGGCTGTTCATTTAGGTTACATGAAGA
AAAACCCCACTATAGGGGCTCATATAAGCGGACAGGACGTAGCGAAAAATAAAGCA
CAATTTATGGAAACAGACAAAGTTCATTTACTATTAGAAGAACTTGCAAAATTTCAT
TCTATATCACGAGCAGTTATCTTTCTAGCTGTCCAGACAGGCATGAGGTTCGAAGAA
ATTATTGCACTAACAAAGAAGGATATTAATTTCACTAAACGTTCAATAACTGTGAAT
AAAGCTTGGGATTACAAGTACACTAATACATTCATTGATACCAAAACAAAAAAATC
ACGAGTGATCTATATTGATAACTCTACCGCTCAATATTTACATTCGTATTTAAATTGG
CATACTGAATATATGAAGGAACATGCTATTAAGAATCCATTGATGTTATTATTCATC
ACTTACCACAATAAGCCAGTAGACAACGCGTCTTGTAATAAAGCTTTGAAGAAGAT
ATGTAGTACAATCAATTCTGAACCAGTGACATTACACAAGCTACGACATACGCATAC
AGGCTTATGTGTAGAAGCGGGTATGGATATTATTTATGTAGCTGATAGGCTTGGTCA
TGATGACATTAATACAACATTAAAATACTATAGTCATCTAAGCTCTAATTTAAGACA
ACATAATCAGTCCAAAGTAGATGCTTTTTTCACACTAAAAACAGATGAAAATACCAC
AAATTTTACCACAAATGCCACAAAAACAACGGAATAAAACGGGTATTATACGATAT
AAAAAAAACTCCAAAACATTCATCCGCCCTTTAATATCAAGGCTTTTCAACGTTTTA
GAGATTTCTTTACATTACTATTTAACGTCCTGAGAGGGATTAACACACACTGATATA
AAGCCATTTAGGATATATATACCACAAATAATACCACAAACATTTTATGTAATAATA
AATATTATTTATTATTACATTGAAATAAATATTCGTTATAAATAGTTTTTATATCAAG
ATGTTTTTTCTCAAGGTTTTTATAAAATGACTTTAATTCTTTTGTTTCAAGTAGTCCAG
AGAAGATTTTTTCAACAGCGTTCTTCTTTCCCTCCACGCATGCCTCTCGCCTGTCCCC
TCAGTTCAGTAATTTCCTGCATTTGCCTGTTTCCAGTCGGTAGATATTCCACAAAACA
GCAGGGAAGCAGCGCTTTTCCGCTGCATAACCCTGCTTCGGGGTCATTATAGCGATT
TTTTCGGTATATCCATCCTTTTTCGCACGATATACAGGATTTTGCCAAAGGGTTCGTG
TAGACTTTCCTTGGTGTATCCAACGGCGTCAGCCGGGCAGGATAGGTGAAGTAGGCC
CACCCGCGAGCGGGTGTTCCTTCTTCACTGTCCCTTATTCGCACCTGGCGGTGCTCAA
CGGGAATCCTGCTCTGCGAGGCTGGCCGGCTACCGCCGGCGTAACAGATGAGGGCA
AGCGGCGGAGAATTACAACTTATATCGTATGGGGCTGACTTCAGGTGCTACATTTGA
AGAGATAAATTGCACTGAAATCTAGAAATATTTTATCTGATTAATAAGATGATCTTC
TTGAGATCGTTTTGGTCTGCGCGTAATCTCTTGCTCTGAAAACGAAAAAACCGCCTT
GCAGGGCGGTTTTTCGAAGGTTCTCTGAGCTACCAACTCTTTGAACCGAGGTAACTG
GCTTGGAGGAGCGCAGTCACCAAAACTTGTCCTTTCAGTTTAGCCTTAACCGGCGCA
TGACTTCAAGACTAACTCCTCTAAATCAATTACCAGTGGCTGCTGCCAGTGGTGCTT
TTGCATGTCTTTCCGGGTTGGACTCAAGACGATAGTTACCGGATAAGGCGCAGCGGT
CGGACTGAACGGGGGGTTCGTGCATACAGTCCAGCTTGGAGCGAACTGCCTACCCG
GAACTGAGTGTCAGGCGTGGAATGAGACAAACGCGGCCATAACAGCGGAATGACAC
CGGTAAACCGAAAGGCAGGAACAGGAGAGCGCACGAGGGAGCCGCCAGGGGGAAA
CGCCTGGTATCTTTATAGTCCTGTCGGGTTTCGCCACCACTGATTTGAGCGTCAGATT
TCGTGATGCTTGTCAGGGGGGCGGAGCCTATGGAAAAACGGCTTTGCCGCGGCCCTC
TCACTTCCCTGTTAAGTATCTTCCTGGCATCTTCCAGGAAATCTCCGCCCCGTTCGTA
AGCCATTTCCGCTCGCCGCAGTCGAACGACCGAGCGTAGCGAGTCAGTGAGCGAGG
AAGCGGAATATATCCTGTATCACATATTCTGCTGACGCACCGGTGCAGCCTTTTTTCT
CCTGCCACATGAAGCACTTCACTGACACCCTCATCAGTGCCAACATAGTAAGCCAGT
ATACACTCCGCTAGCGCTGATGTCCGGCGGTGCTTTTGCCGTTACGCACCACCCCGT
CAGTAGCTGAACAGGAGGGACAGCTGATAGAAACAGAAGCCACTGGAGCACCTCA
AAAACACCATCATACACTAAATCAGTAAG
hly 5′ UTR (SEQ ID NO: 11)
GATAAAGCAAGCATATAATATTGCGTGTAGAAGGAGAGTGAAACCC

1. A genetically engineered Listeria monocytogenes bacterium comprising a nucleic acid sequence encoding interleukin 2 (IL-2), interleukin 12 (IL-12), or Cluster of Differentiation 40 ligand (CD40L).

2. The bacterium of claim 1, wherein the nucleic acid sequence encodes IL-2.

3. The bacterium of claim 1, wherein the nucleic acid sequence encodes IL-12.

4. The bacterium of claim 1, wherein the nucleic acid sequence encodes CD40L.

5. The bacterium of claim 1, wherein the nucleic acid sequence further encodes a Listeria monocytogenes secretion signal.

6. The bacterium of claim 1, wherein the Listeria monocytogenes secretion signal comprises an ActA secretion signal.

7. The bacterium of claim 6, wherein the ActA secretion signal comprises amino acids 1 to 30 of the ActA protein or amino acids 1 to 100 of the ActA protein.

8. The bacterium of claim 7, wherein the ActA secretion signal comprises amino acids 1-100 of the ActA protein.

9. The bacterium of claim 5, wherein the Listeria monocytogenes secretion signal comprises the amino acid sequence set forth in SEQ ID NO: 9.

10. The bacterium of claim 1, wherein the nucleic acid sequence further comprises a 5′ untranslated region.

11. The bacterium of claim 10, wherein the 5′ untranslated region comprises the 5′ untranslated region of an hly gene.

12. The bacterium of claim 10, wherein the 5′ untranslated region comprises the nucleotide sequence set forth in SEQ ID NO: 11.

13. The bacterium of claim 1, wherein the nucleic acid sequence comprises SEQ ID NO: 10.

14. The bacterium of claim 1, wherein the nucleic acid sequence is integrated into the bacterial genome.

15. The bacterium of claim 14, wherein the nucleic acid sequence is integrated into the bacterial genome at the attBB′ site in the tRNA^{Arg} locus.

16.-18. (canceled)

19. A method of treating a cancer in a subject in need thereof comprising administering to the subject a composition comprising a genetically engineered Listeria monocytogenes bacterium comprising a nucleic acid sequence encoding interleukin 2 (IL-2), interleukin 12 (IL-12), or Cluster of Differentiation 40 ligand (CD40L).

20. The method of claim 19, wherein the cancer comprises melanoma or neuroblastoma.

21. The method of claim 19, wherein the cancer comprises one or more solid tumors.

22. The method of claim 20, wherein the genetically engineered Listeria monocytogenes bacterium persists in the one or more solid tumors for about 1 day to about 3 weeks post administration.

23. The method of claim 19, wherein the method is effective for reducing tumor volume or slowing tumor growth, and wherein the composition is administered intravenously or intratumorally.

24.-57. (canceled)


SEQUENCE LISTING (11 sequences)

SEQ ID NO: 1 - DNA, 523 bp, synthetic construct
  source 1..523: mol_type = other DNA
aggagagtgaaacccatgggtgggattaaatagatttatgcgtgcgatgatggtagttttcattactgccaactgcattacgattaaccccgacataatatttgcagcgccaacatcttcttccactaagaagactcagctacaacttgaacaccttctgttggacctacaaatgattctaaatggaataaacaattataaaaatcctaaattaactagaatgctcacgttcaaattctacatgccgaaaaaagcaaccgagttaaaacatttacaatgtcttgaagaagaacttaaaccattggaagaagtgcttaacttggcccaaagtaaaaattttcatttacgtccacgagatttaattagcaatatcaatgtcattgtattagaattaaaaggtagtgaaaccacatttatgtgcgaatatgctgatgagacagcgacaattgttgaatttttaaaccgctggattacattttgtcaatcaattatctcgacgttaacgtgagtcgacctcgagggg

SEQ ID NO: 2 - DNA, 744 bp, synthetic construct
  source 1..744: mol_type = other DNA
gaaggagagtgaaacccatgggtgggattaaatagatttatgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtggatccccaacatcttcttccactaagaagactcagctacaacttgaacaccttctgttggacctacaaatgattctaaatggaataaacaattataaaaatcctaaattaactagaatgctcacgttcaaattctacatgccgaaaaaagcaaccgagttaaaacatttacaatgtcttgaagaagaacttaaaccattggaagaagtgcttaacttggcccaaagtaaaaattttcatttacgtccacgagatttaattagcaatatcaatgtcattgtattagaattaaaaggtagtgaaaccacatttatgtgcgaatatgctgatgagacagcgacaattgttgaatttttaaaccgctggattacattttgtcaatcaattatctcgacgttaacgtgagtcgacctcgaggggggg

SEQ ID NO: 3 - DNA, 44 bp, synthetic construct
  source 1..44: mol_type = other DNA
agggaacaaaagctgggtacccattatgctttggcagtttattc

SEQ ID NO: 4 - DNA, 30 bp, synthetic construct
  source 1..30: mol_type = other DNA
agcctgacatgggtttcactctccttctac

SEQ ID NO: 5 - DNA, 22 bp, synthetic construct
  source 1..22: mol_type = other DNA
tcaaccagatgttctccctgta

SEQ ID NO: 6 - DNA, 22 bp, synthetic construct
  source 1..22: mol_type = other DNA
cactgtaagccatttcgtcatc

SEQ ID NO: 7 - DNA, 5996 bp, synthetic construct
  source 1..5996: mol_type = other DNA
ttggcagcatcacccgacgcactttgcgccgaataaatacctgtgacggaagatcacttcgcagaataaataaatcctggtgtccctgttgataccgggaagccctgggacgtcgggccctttcgtcttcaagaattaattcccaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattggggatcggaattcgagctccattatgctttggcagtttattcttgacatgtagtgagggggctggtataatcacatacggccgataaagcaagcatataatattgcgtttcatctttagaagcgaatttcgccaatattataattatcaaaagagaggggtggcaaacggtatttggcattattaggttaaaaaatgtagaaggagagtgaaacccatggaaaaggatcccccgggctgcaggaattcgatatcaagcttatcgataccgtcgacctcgagggggggcccggtacccagcttttgttccctttagtgagggttaattcccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttaactacaagctttttagacatctaatcttttctgaagtacatccgcaactgtccatactctgatgtttcatatgatcatcataattctgtctcattatataacatcctccataccttctattatagaataccataaactcatctggcaattcatttcgagtcacgaagaacggaaaaactgccggtttttatattacaaatgtattaagtttttctattaacaaaaaacaataggtttcccatagcgaaagttgttgattaacgttcacatcccacttacactataaaggtttacccagcaatacatctcaagccctaagaatacacgttcgcttttcaactgttacagaattattacaaatagttggtatagtcctctttagcctttggagctattatctcatcatttgttttttaggtgaaaactgggtaaacttagtattaatcaatataaaattaattctcaaatacttaattacgtactgggattttctgaaaaaaagatctccaaaaataaacaggtggtggtattaatgaagataaaaaaattagcaaacggtaaatattgtgttcgcctacgtataaaagtcgatggtgaatggaaagaaaagcgtttgacagatacaagtgaaacaaacttaatgtataaagcatctaaattattaaaacaagttcagcatgatagtagttctctgaaagaatggaacttcaaagaattttatacgctattcatgaaaacatttaaagatgggaaaagtagtcaatctactattaatttatacgatcttgcttataatcaattcgttgattatttcgatgaaaaaattaaatttaattcgattgatgcggttcaatatcaacaatttattaatcatttatctgtagactatgcaatatccactgtagacaccagacaccgcaaaattagagcgatttttaacaaggctgttcatttaggttacatgaagaaaaaccccactataggggctcatataagcggacaggacgtagcgaaaaataaagcacaatttatggaaacagacaaagttcatttactattagaagaacttgcaaaatttcattctatatcacgagcagttatctttctagctgtccagacaggcatgaggttcgaagaaattattgcactaacaaagaaggatattaatttcactaaacgttcaataactgtgaataaagcttgggattacaagtacactaatacattcattgataccaaaacaaaaaaatcacgagtgatctatattgataactctaccgctcaatatttacattcgtatttaaattggcatactgaatatatgaaggaacatgctattaagaatccattgatgttattattcatcacttaccacaataagccagtagacaacgcgtcttgtaataaagctttgaagaagatatgtagtacaatcaattctgaaccagtgacattacacaagctacgacatacgcatacaggcttatgtgtagaagcgggtatggatattatttatgtagctgataggcttggtcatgatgacattaatacaacattaaaatactatagtcatctaagctctaatttaagacaacataatcagtccaaagtagatgcttttttcacactaaaaacagatgaaaataccacaaattttaccacaaatgccacaaaaacaacggaataaaacgggtattatacgatataaaaaaaactccaaaacattcatccgccctttaatatcaaggcttttcaacgttttagagatttctttacattactatttaacgtcctgagagggattaacacacactgatataaagccatttaggatatatataccacaaataataccacaaacattttatgtaataataaatattatttattattacattgaaataaatattcgttataaatagtttttatatcaagatgttttttctcaaggtttttataaaatgactttaattcttttgtttcaagtagtccagagaagattttttcaacagcgttcttctttccctccacgcatgcctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggcggagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaag

SEQ ID NO: 8 - DNA, 6436 bp, synthetic construct
  source 1..6436: mol_type = other DNA
ttggcagcatcacccgacgcactttgcgccgaataaatacctgtgacggaagatcacttcgcagaataaataaatcctggtgtccctgttgataccgggaagccctgggacgtcgggccctttcgtcttcaagaattaattcccaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattggggatcggaattcgagctccattatgctttggcagtttattcttgacatgtagtgagggggctggtataatcacatacggccgataaagcaagcatataatattgcgtttcatctttagaagcgaatttcgccaatattataattatcaaaagagaggggtggcaaacggtatttggcattattaggttaaaaaatgtagaaggagagtgaaacccatgggtgggattaaatagatttatgcgtgcgatgatggtagttttcattactgccaactgcattacgattaaccccgacataatatttgcagcgccaacatcttcttccactaagaagactcagctacaacttgaacaccttctgttggacctacaaatgattctaaatggaataaacaattataaaaatcctaaattaactagaatgctcacgttcaaattctacatgccgaaaaaagcaaccgagttaaaacatttacaatgtcttgaagaagaacttaaaccattggaagaagtgcttaacttggcccaaagtaaaaattttcatttacgtccacgagatttaattagcaatatcaatgtcattgtattagaattaaaaggtagtgaaaccacatttatgtgcgaatatgctgatgagacagcgacaattgttgaatttttaaaccgctggattacattttgtcaatcaattatctcgacgttaacgtgagtcgacctcgagggggggcccggtacccagcttttgttccctttagtgagggttaattcccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttaactacaagctttttagacatctaatcttttctgaagtacatccgcaactgtccatactctgatgtttcatatgatcatcataattctgtctcattatataacatcctccataccttctattatagaataccataaactcatctggcaattcatttcgagtcacgaagaacggaaaaactgccggtttttatattacaaatgtattaagtttttctattaacaaaaaacaataggtttcccatagcgaaagttgttgattaacgttcacatcccacttacactataaaggtttacccagcaatacatctcaagccctaagaatacacgttcgcttttcaactgttacagaattattacaaatagttggtatagtcctctttagcctttggagctattatctcatcatttgttttttaggtgaaaactgggtaaacttagtattaatcaatataaaattaattctcaaatacttaattacgtactgggattttctgaaaaaaagatctccaaaaataaacaggtggtggtattaatgaagataaaaaaattagcaaacggtaaatattgtgttcgcctacgtataaaagtcgatggtgaatggaaagaaaagcgtttgacagatacaagtgaaacaaacttaatgtataaagcatctaaattattaaaacaagttcagcatgatagtagttctctgaaagaatggaacttcaaagaattttatacgctattcatgaaaacatttaaagatgggaaaagtagtcaatctactattaatttatacgatcttgcttataatcaattcgttgattatttcgatgaaaaaattaaatttaattcgattgatgcggttcaatatcaacaatttattaatcatttatctgtagactatgcaatatccactgtagacaccagacaccgcaaaattagagcgatttttaacaaggctgttcatttaggttacatgaagaaaaaccccactataggggctcatataagcggacaggacgtagcgaaaaataaagcacaatttatggaaacagacaaagttcatttactattagaagaacttgcaaaatttcattctatatcacgagcagttatctttctagctgtccagacaggcatgaggttcgaagaaattattgcactaacaaagaaggatattaatttcactaaacgttcaataactgtgaataaagcttgggattacaagtacactaatacattcattgataccaaaacaaaaaaatcacgagtgatctatattgataactctaccgctcaatatttacattcgtatttaaattggcatactgaatatatgaaggaacatgctattaagaatccattgatgttattattcatcacttaccacaataagccagtagacaacgcgtcttgtaataaagctttgaagaagatatgtagtacaatcaattctgaaccagtgacattacacaagctacgacatacgcatacaggcttatgtgtagaagcgggtatggatattatttatgtagctgataggcttggtcatgatgacattaatacaacattaaaatactatagtcatctaagctctaatttaagacaacataatcagtccaaagtagatgcttttttcacactaaaaacagatgaaaataccacaaattttaccacaaatgccacaaaaacaacggaataaaacgggtattatacgatataaaaaaaactccaaaacattcatccgccctttaatatcaaggcttttcaacgttttagagatttctttacattactatttaacgtcctgagagggattaacacacactgatataaagccatttaggatatatataccacaaataataccacaaacattttatgtaataataaatattatttattattacattgaaataaatattcgttataaatagtttttatatcaagatgttttttctcaaggtttttataaaatgactttaattcttttgtttcaagtagtccagagaagattttttcaacagcgttcttctttccctccacgcatgcctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggcggagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaag

SEQ ID NO: 9 - DNA, 6652 bp, synthetic construct
  source 1..6652: mol_type = other DNA
ttggcagcatcacccgacgcactttgcgccgaataaatacctgtgacggaagatcacttcgcagaataaataaatcctggtgtccctgttgataccgggaagccctgggacgtcgggccctttcgtcttcaagaattaattcccaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattggggatcggaattcgagctccattatgctttggcagtttattcttgacatgtagtgagggggctggtataatcacatacggccgataaagcaagcatataatattgcgtttcatctttagaagcgaatttcgccaatattataattatcaaaagagaggggtggcaaacggtatttggcattattaggttaaaaaatgtagaaggagagtgaaacccatgggtgggattaaatagatttatgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtggatccccaacatcttcttccactaagaagactcagctacaacttgaacaccttctgttggacctacaaatgattctaaatggaataaacaattataaaaatcctaaattaactagaatgctcacgttcaaattctacatgccgaaaaaagcaaccgagttaaaacatttacaatgtcttgaagaagaacttaaaccattggaagaagtgcttaacttggcccaaagtaaaaattttcatttacgtccacgagatttaattagcaatatcaatgtcattgtattagaattaaaaggtagtgaaaccacatttatgtgcgaatatgctgatgagacagcgacaattgttgaatttttaaaccgctggattacattttgtcaatcaattatctcgacgttaacgtgagtcgacctcgagggggggcccggtacccagcttttgttccctttagtgagggttaattcccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttaactacaagctttttagacatctaatcttttctgaagtacatccgcaactgtccatactctgatgtttcatatgatcatcataattctgtctcattatataacatcctccataccttctattatagaataccataaactcatctggcaattcatttcgagtcacgaagaacggaaaaactgccggtttttatattacaaatgtattaagtttttctattaacaaaaaacaataggtttcccatagcgaaagttgttgattaacgttcacatcccacttacactataaaggtttacccagcaatacatctcaagccctaagaatacacgttcgcttttcaactgttacagaattattacaaatagttggtatagtcctctttagcctttggagctattatctcatcatttgttttttaggtgaaaactgggtaaacttagtattaatcaatataaaattaattctcaaatacttaattacgtactgggattttctgaaaaaaagatctccaaaaataaacaggtggtggtattaatgaagataaaaaaattagcaaacggtaaatattgtgttcgcctacgtataaaagtcgatggtgaatggaaagaaaagcgtttgacagatacaagtgaaacaaacttaatgtataaagcatctaaattattaaaacaagttcagcatgatagtagttctctgaaagaatggaacttcaaagaattttatacgctattcatgaaaacatttaaagatgggaaaagtagtcaatctactattaatttatacgatcttgcttataatcaattcgttgattatttcgatgaaaaaattaaatttaattcgattgatgcggttcaatatcaacaatttattaatcatttatctgtagactatgcaatatccactgtagacaccagacaccgcaaaattagagcgatttttaacaaggctgttcatttaggttacatgaagaaaaaccccactataggggctcatataagcggacaggacgtagcgaaaaataaagcacaatttatggaaacagacaaagttcatttactattagaagaacttgcaaaatttcattctatatcacgagcagttatctttctagctgtccagacaggcatgaggttcgaagaaattattgcactaacaaagaaggatattaatttcactaaacgttcaataactgtgaataaagcttgggattacaagtacactaatacattcattgataccaaaacaaaaaaatcacgagtgatctatattgataactctaccgctcaatatttacattcgtatttaaattggcatactgaatatatgaaggaacatgctattaagaatccattgatgttattattcatcacttaccacaataagccagtagacaacgcgtcttgtaataaagctttgaagaagatatgtagtacaatcaattctgaaccagtgacattacacaagctacgacatacgcatacaggcttatgtgtagaagcgggtatggatattatttatgtagctgataggcttggtcatgatgacattaatacaacattaaaatactatagtcatctaagctctaatttaagacaacataatcagtccaaagtagatgcttttttcacactaaaaacagatgaaaataccacaaattttaccacaaatgccacaaaaacaacggaataaaacgggtattatacgatataaaaaaaactccaaaacattcatccgccctttaatatcaaggcttttcaacgttttagagatttctttacattactatttaacgtcctgagagggattaacacacactgatataaagccatttaggatatatataccacaaataataccacaaacattttatgtaataataaatattatttattattacattgaaataaatattcgttataaatagtttttatatcaagatgttttttctcaaggtttttataaaatgactttaattcttttgtttcaagtagtccagagaagattttttcaacagcgttcttctttccctccacgcatgcctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggcggagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaag

SEQ ID NO: 10 - DNA, 6697 bp, synthetic construct
  source 1..6697: mol_type = other DNA
ttggcagcatcacccgacgcactttgcgccgaataaatacctgtgacggaagatcacttcgcagaataaataaatcctggtgtccctgttgataccgggaagccctgggacgtcgggccctttcgtcttcaagaattaattcccaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattaattccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggaattggggatcggaattcgagctccattatgctttggcagtttattcttgacatgtagtgagggggctggtataatcacatacggccgataaagcaagcatataatattgcgtttcatctttagaagcgaatttcgccaatattataattatcaaaagagaggggtggcaaacggtatttggcattattaggttaaaaaatgtagaaggagagtgaaacccatggataaagcaagcatataatattgcgtgtagaaggagagtgaaacccgtgggattaaatagatttatgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtggatccccaacatcttcttccactaagaagactcagctacaacttgaacaccttctgttggacctacaaatgattctaaatggaataaacaattataaaaatcctaaattaactagaatgctcacgttcaaattctacatgccgaaaaaagcaaccgagttaaaacatttacaatgtcttgaagaagaacttaaaccattggaagaagtgcttaacttggcccaaagtaaaaattttcatttacgtccacgagatttaattagcaatatcaatgtcattgtattagaattaaaaggtagtgaaaccacatttatgtgcgaatatgctgatgagacagcgacaattgttgaatttttaaaccgctggattacattttgtcaatcaattatctcgacgttaacgtgagtcgacctcgagggggggcccggtacccagcttttgttccctttagtgagggttaattcccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatttagatgtctaaaaagctttaactacaagctttttagacatctaatcttttctgaagtacatccgcaactgtccatactctgatgtttcatatgatcatcataattctgtctcattatataacatcctccataccttctattatagaataccataaactcatctggcaattcatttcgagtcacgaagaacggaaaaactgccggtttttatattacaaatgtattaagtttttctattaacaaaaaacaataggtttcccatagcgaaagttgttgattaacgttcacatcccacttacactataaaggtttacccagcaatacatctcaagccctaagaatacacgttcgcttttcaactgttacagaattattacaaatagttggtatagtcctctttagcctttggagctattatctcatcatttgttttttaggtgaaaactgggtaaacttagtattaatcaatataaaattaattctcaaatacttaattacgtactgggattttctgaaaaaaagatctccaaaaataaacaggtggtggtattaatgaagataaaaaaattagcaaacggtaaatattgtgttcgcctacgtataaaagtcgatggtgaatggaaagaaaagcgtttgacagatacaagtgaaacaaacttaatgtataaagcatctaaattattaaaacaagttcagcatgatagtagttctctgaaagaatggaacttcaaagaattttatacgctattcatgaaaacatttaaagatgggaaaagtagtcaatctactattaatttatacgatcttgcttataatcaattcgttgattatttcgatgaaaaaattaaatttaattcgattgatgcggttcaatatcaacaatttattaatcatttatctgtagactatgcaatatccactgtagacaccagacaccgcaaaattagagcgatttttaacaaggctgttcatttaggttacatgaagaaaaaccccactataggggctcatataagcggacaggacgtagcgaaaaataaagcacaatttatggaaacagacaaagttcatttactattagaagaacttgcaaaatttcattctatatcacgagcagttatctttctagctgtccagacaggcatgaggttcgaagaaattattgcactaacaaagaaggatattaatttcactaaacgttcaataactgtgaataaagcttgggattacaagtacactaatacattcattgataccaaaacaaaaaaatcacgagtgatctatattgataactctaccgctcaatatttacattcgtatttaaattggcatactgaatatatgaaggaacatgctattaagaatccattgatgttattattcatcacttaccacaataagccagtagacaacgcgtcttgtaataaagctttgaagaagatatgtagtacaatcaattctgaaccagtgacattacacaagctacgacatacgcatacaggcttatgtgtagaagcgggtatggatattatttatgtagctgataggcttggtcatgatgacattaatacaacattaaaatactatagtcatctaagctctaatttaagacaacataatcagtccaaagtagatgcttttttcacactaaaaacagatgaaaataccacaaattttaccacaaatgccacaaaaacaacggaataaaacgggtattatacgatataaaaaaaactccaaaacattcatccgccctttaatatcaaggcttttcaacgttttagagatttctttacattactatttaacgtcctgagagggattaacacacactgatataaagccatttaggatatatataccacaaataataccacaaacattttatgtaataataaatattatttattattacattgaaataaatattcgttataaatagtttttatatcaagatgttttttctcaaggtttttataaaatgactttaattcttttgtttcaagtagtccagagaagattttttcaacagcgttcttctttccctccacgcatgcctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggcggagaattacaacttatatcgtatggggctgacttcaggtgctacatttgaagagataaattgcactgaaatctagaaatattttatctgattaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgatgtccggcggtgcttttgccgttacgcaccaccccgtcagtagctgaacaggagggacagctgatagaaacagaagccactggagcacctcaaaaacaccatcatacactaaatcagtaag

SEQ ID NO: 11 - DNA, 46 bp, synthetic construct
  source 1..46: mol_type = other DNA
gataaagcaagcatataatattgcgtgtagaaggagagtgaaaccc